(19) United States
(12) Patent Application Publication
(10) Pub. No.: US 2010/0298547 A1
(43) Pub. Date: Nov. 25, 2010

(54) METHODS FOR THE DIRECTED EXPANSION OF EPITOPES FOR USE AS ANTIBODY LIGANDS

(76) Inventor: Dustan Bonnin, Belmont, MA (US)

Correspondence Address: ROPES & GRAY LLP, PATENT DOCKETING 39/41, ONE INTERNATIONAL PLACE, BOSTON, MA, 02110-2624, US

(21) Appl. No.: 12/451,323

(22) PCT Filed: May 7, 2008
(86) PCT No.: PCT/US2008/005919

§ 371 Date: Apr. 16, 2010

Related U.S. Application Data
(60) Provisional application No. 60/928,225, filed on May 7, 2007.
(60) Provisional application No. 60/999,283, filed on Oct. 16, 2007.
(60) Provisional application No. 60/999,284, filed on Oct. 16, 2007.
(60) Provisional application No. 61/124,689, filed on Apr. 17, 2008.

(51) Int. Cl. C07K 16/00; C40B 30/04

(52) U.S. Cl. 530/388.1; 506/9

(57) ABSTRACT

The instant invention comprises a process for selecting and manufacturing antibodies useful for therapeutic, prophylactic, diagnostic or research purposes using epitope peptide mixtures synthesized by the solid phase synthesis, such process defined by a set of rules regarding the identity and the frequency of occurrence of amino acids that substitute a base or native amino acid of a known epitope. The resulting antibodies are related to but distinct from antibodies that bind to the known epitope.

### RELATED APPLICATION

This application claims the benefit of U.S. Provisional Application 60/928,225, filed May 7, 2007, U.S. Provisional Application 60/999,283, filed Oct. 16, 2007, U.S. Provisional Application 60/999,284, filed Oct. 16, 2007, and U.S. Provisional Application 61/124,689, filed Apr. 17, 2008. This application is also a continuation-in-part of U.S. application Ser. No. 11/787,229, filed Apr. 13, 2007, which claims the benefit of U.S. Provisional Application 60/792,085, filed Apr. 13, 2006. The disclosure in all of the above-listed applications is incorporated herein by reference in its entirety.

### BACKGROUND OF THE INVENTION

In the recent years, antibodies have cemented their position as a class of effective therapeutic agents against various diseases and conditions. Currently there are 21 approved antibody-based drugs in the world, and a number of them are in the pipeline.

A single antibody may be specific for one antigen, or may recognize multiple antigens (Notkins, A. L. et al., Curr. Topics Microbiol. Immunol. 252:241, 2000; De Ciechi, P A et al., Mol. Divers. 1:79, 1995). However, an antibody binding to a relevant epitope does not ensure therapeutic effectiveness. An antibody may bind with a wide range of binding affinity, and the small differences in the binding configuration may cause conformational changes in the target protein, may control the degree with which the antibody competes with other antibodies or binding partners of the target, or may trigger varied responses from the immune cells that recognize the antigen-antibody complex.

To further complicate the matter, in diseases such as an autoimmune disease or in conditions such as transplant rejection where the endogenous antibodies amount misguided attack on sells tissues and organs, an identified epitope that at one stage of a disease is a viable target does not stay so. This phenomenon, epitope spreading, reduces the efficacy of a therapeutic or prophylactic antibody that compete with and interfere the binding of the offending endogenous antibodies, because the body starts recognizing the portions of the target protein adjoining the initial epitope as a new epitope. (N. Suciu-Foca et al., Immunol. Rev. 1998, 164:241).

Another obstacle to preparing therapeutically useful antibodies is lowly immunogenic peptides and epitopes. Therapeutically useful antibodies may not easily arise or be identified against antigens and epitopes that do not elicit strong immune responses. Such a phenomenon has been long recognized in the field of vaccination and immune enhancing treatments against invading pathogens or against cancers, especially against invading pathogens practicing immune evasion. Further discussed below is the enhancement of immune reactivity, either for efficient antibody production or for effective vaccination therapy.

Immunization programs in the effort to control infectious diseases, such as small-pox, polio, measles, mumps, rubella, Haemophilus influenza, pertussis, tetanus, and diphtheria, used centuries old technology to safely create an immune response in a host prior to pathogenic infection by the live organism. These vaccination protocols called for the introduction to the host of an inactive form of the actual pathogen, and in so doing an active immune response was created. While improvements to the techniques have been made in the form of differing types of inactivation of pathogen or in the use of adjuvants to enhance immunogenicity, a need remains in the development of vaccines that can handle the infectious agents such as human immunodeficiency virus (HIV), cytomegalovirus, and severe acute respiratory syndrome coronavirus, as well as bacteria such as Pseudomonas aeruginosa, Neisseria gonorrhea, or Mycobacterium tuberculosis or parasitic diseases such as malaria or hookworm disease that are generally refractive to traditional vaccine therapies.

These infectious agents, bacteria, and parasitic diseases are harder to treat using the inactive pathogen vaccine approach because of the organism's ability to evade host detection by “immune evasion”. The HIV or the flu virus has the ability to alter its immune profile multiple times in the amount of time less than a calendar year progressively marginalizing the effectiveness of the even the most recently created vaccine.

Advances in eliciting stronger immune responses have been made with the development of antigen/epitope non-specific treatments that boost immune activity, such as the adjuvant alum (see Vaccine Adjuvants and Delivery Systems, edited by Manmohan Singh 2007 Wiley & Sons ISBN: 978-0-471-73907-4, incorporated by reference herein, for an extensive review of vaccine adjuvants), as well as development of immunogens based on the understanding of the genetic basis of these pathogens (GenBank, a database managed by the National Center for Biotechnology Information, now has over 85 billion base pairs in its database. Searching based on pathogen is widely used http://www.ncbi.nlm.nih.gov/Genbank/). The latter has opened up the possibility of utilizing discrete sequences of proteins derived from the pathogen as immunizing agents. These peptide-based vaccines are antigenic determinant-specific, intended to boost immune reactivity, and are administered using methods designed to excite immune function.

Therapeutic antibodies can be highly specific and effective, but the means to create them and to identify therapeutically active species are still laborious and expensive.

The generation of antibodies is well known in the art. Antibodies are synthesized by B cells in response to a B cell Receptor (BCR) interacting with a recognized ligand. Prior to the introduction of what is perceived as an antigen by an organism, a variety of antibodies, some of which bind the innoculant, persist in a host in the background of the active immune system. When the antigen is introduced into the organism, these preexisting antibodies (germline antibodies) interact with the antigen, and initiates a cascade of B cell proliferation events which result in antibodies with a higher affinity for the antigen via a process termed affinity maturation. In this natural machinery, multiple antibodies that bind to the introduced antigen, with a variety of binding region sequences, are produced, each clonal B cell line producing a particular antibody.

In order to create and produce antibodies for specific target antigens, various methods have been developed. The art found advantages to produce a quantity of antibodies with identical binding regions (monoclonal antibodies). One well-known method to generate monoclonal antibodies is the generation of hybridomas. Briefly, a hybridoma is generated by fusing a murine B cell with a murine tumor cell. The resulting combination is an immortal cell line that is not dependent upon constant stimulation, which produces the desired antibodies. The homogenicity of a hybridoma line makes the system attractive to produce a highly defined drug product for clinical administration. However, hybridomas have the large downside in a clinical setting of being mouse-derived, as the human immune system recognizes mouse antibodies as being foreign, thus clearing them from the system.

To overcome this limitation, “humanized antibodies” are created. Humanized antibodies, created by genetic engineering, possess the variable regions of the mouse antibodies from hybridomas and the rest of the immunoglobulin, for example the constant region, derive from the human immunoglobulin.

However, human immune systems recognized even these humanized antibodies as foreign proteins; the complimentary determining regions (CDR) that provide the antigen specificity of the antibody if derived from a mouse causes an immune reaction in a human. To further improve the antibodies, transgenic mice which produce fully human antibodies in the context of the mouse immune system have been created (Mendez M J et al Nature Genetics 15:146, 1997). Alternatively, adopted from expression phage library technology, expression systems based on filamentous bacteriophages like M13 were created to present human antibody gene products. Briefly, to identify the desired antibodies, phages expressing various human antibodies are contacted with an antigen or protein of interest. The phages expressing irrelevant antibodies do not bind to the antigen, and are washed away. The antibody sequence is retrieved from the bound phage and cloned into expression systems such as Escherichia coli cells, for example, for antibody production.

Despite the improvements, production of antibodies in large quantities remains challenging: how to produce the required amount. Further, these systems are both dependent on the quality of antigens. However, the use of antigens purified from naïve material is limited due to logistic and cost barriers. It has been reported that the amount of effort required to produce antigen equals or exceeds the effort required for the antibody selection process (Hust and Duebel, Trends in Biotechnology 22:8, 2004).

There is therefore a need for an improved method of identifying and producing antibodies, including improving the means and the cost to obtain appropriate, immunogenic antigens, that are therapeutically useful.

### SUMMARY OF THE INVENTION

Methods currently in the art to identify a therapeutic antibody include high-throughput screening of an antibody library for binding to an epitope in the hope of identifying a prototype antibody, followed by mostly random point mutations of the variable region sequence to create candidates the binding characteristics of which would be different from the prototype, thus exhibiting a physiological effect different from the prototype.

The instant invention comprises an improved process for producing antibodies that are therapeutically or prophylactically useful, or useful for use as research reagents, as diagnostic tools, as means to interrogate species differences in protein sequence, or as a means to overcome problems related to species differences in protein sequence. The method is drawn to increasing the diversity of antibodies generated to react with a ligand. Further, the method is drawn to overcoming the problem of creating antibodies against ligands with low immunogenicity. Still further, the method is drawn to overcoming problems relating to generating antibodies having reactivities to only a single species. The instant invention comprises a method of creating antibody reagents for use in research studies. The instant invention comprises a method of creating antibody reagents for use as diagnostic tools. The instant invention further comprises a method for the generation of antibodies useful as therapeutic agents for the treatment of disease. Using the same principle, antibodies may be produced in vivo, i.e., the compositions for stimulating antibody production may be used as vaccines. Immunization steps of all the representative methods described below can be modified for in vivo use of the immunogens of the present invention as vaccines.

The method of the instant invention also encompasses an augmentation of the paratopes associated with an antibody response to an antigen of interest. The method of the instant invention further encompasses the generation of novel functioning antibodies having antigen binding properties that elicit a varied amount of downstream consequences to the binding event.

Briefly, the method comprises the steps of selecting a protein of interest, determining relevant epitopes within the protein, selecting the relevant epitope, performing directed permutations of the epitope so as to create an expanded yet related series of antigens, performing solid phase synthesis thus creating a directed sequence polymer (DSP), using the DSP collectively as a set of antigens by placing the DSP in contact with a means of antibody generation, determining the activity of the generated antibodies, selecting antibodies having the desired activity, and utilizing the antibody as a single species reagent, multi-species reagent, single species diagnostic, multi-species diagnostic, or alternatively as a therapeutic. The means of antibody generation is, for example, an animal to be immunized by the DSP and cells from such an animal (e.g. spleen cells from a mouse for monoclonal antibody production), a phage display library, or a B cell library.

A preferred method of the instant invention comprises the steps of selecting a protein, either having no known function, having a known or anticipated research interest, having a known or anticipated diagnostic interest, or disease association, selecting an epitope within the protein, which epitope may have a range of immunogenicity, from no known immunogenicity to being weakly immunogenic to being strongly immunogenic, performing directed permutations of the epitope based on a set of rules that govern the ratios of from one to three amino acid substitutions plus an alanine substitution, synthesizing the DSP using solid phase chemistry, creating antibodies by introducing the DSP into an in vivo setting, or alternatively introducing the DSP into an in vitro setting, or still alternatively contacting the DSP with a system of maintaining the connection between antibody phenotype and genotype such as phage display, determining the activity of the generated antibodies by contacting the antibodies with the native molecule of interest, selecting antibodies having desired activity, such activity being either of a higher affinity antibody, or alternatively a lower affinity antibody, a single species reactivity, or alternatively a multi-species reactivity, a single-molecule of interest reactivity or alternatively a multi-molecule reactivity. In certain embodiments, the desired activity is antagonistic to the activity of the target, and in certain preferred embodiments, the desired activity is blocking the activity of the target. In other embodiments, the desired activity is agonistic to the activity of the target protein. In certain embodiments the desired characteristic of antibodies is so that they are useful as a reagent, or diagnostic, or alternatively as a therapeutic. In further embodiments, antibodies with multiple characteristics are combined into a single reagent, diagnostic, or therapeutic. In further embodiments, said multiple characterisitics comprise angonist, antagonist, or null activities to the target protein.

Alternatively, a method of the instant invention comprises selecting a protein of interest known to have a discontinuous epitope, selecting the amino acids that make up the epitope, combining the amino acids into a linear peptide to performing directed permutations to create the DSP and developing antibodies as above.

Yet other embodiments of the instant invention comprises selecting two or more proteins of interest, two or more epitopes are selected with at least one epitope deriving from each protein of interest, combining the epitopes into a linear sequence to performing directed permutations to create the DSP and developing antibodies as above.

Alternatively, the instant invention encompasses methods of producing antibodies, the methods comprising: selecting a protein of interest, selecting the amino acids that make up the epitope, combining the amino acids into a linear peptide, performing directed permutations, synthesizing the DSP using solid phase chemistry, preparing the DSP as a pharmaceutically acceptable salt, introducing the DSP into a host, harvesting primary tissue containing antibody from the host after one week, alternatively harvesting primary tissue containing antibody from the host after a time greater one week, determining the activity of the generated antibodies, selecting, and utilizing the antibody as a reagent, diagnostic, or alternatively as a therapeutic.

Alternatively, the instant invention encompasses methods of producing antibodies, the methods comprising: selecting a protein of interest, selecting a first species, selecting further species, selecting the amino acids that make up the epitope, determining the species differences in the epitope, combining the amino acids into a linear peptide, performing directed permutations using the species differences as the rules for permutation, synthesizing the DSP using solid phase chemistry, preparing the DSP as a pharmaceutically acceptable salt, introducing the DSP into a host that is the same as one of the species who's sequences makes up the rules for the DSP, alternatively, introducing the DSP into a host that is different than any of the species whose sequences make up the rules for the DSP, harvesting primary tissue containing antibody from the host after one week, alternatively harvesting primary tissue containing antibody from the host after a time greater one week, determining the activity of the generated antibodies, selecting, and utilizing the antibody as a reagent, diagnostic, or alternatively as a therapeutic.

Alternatively, the instant invention encompasses methods of producing antibodies, the methods comprising: selecting a protein of interest, selecting a first species, selecting further species, selecting the amino acids that make up the epitope, determining the species differences in the epitope, combining the amino acids into a linear peptide, performing directed permutations using the species differences as the rules for permutation, synthesizing the DSP using solid phase chemistry, preparing the DSP as a pharmaceutically acceptable salt, introducing the DSP into a host that is the same as one of the species who's sequences makes up the rules for the DSP, alternatively, introducing the DSP into a host that is different than any of the species who's sequences make up the rules for the DSP, harvesting primary tissue containing antibody generating cells from the host after one week, alternatively harvesting primary tissue containing antibody generating cells from the host after a time greater one week, determining the activity of the generated antibodies, correlating the activity of an antibody to the genes inside the cells that produced the antibody, selecting, and utilizing the antibody as a reagent, diagnostic, or alternatively as a therapeutic.

Alternatively, the instant invention encompasses methods of producing antibodies, the methods comprising: selecting a protein of interest known to have a discontinuous epitope, selecting the amino acids that make up the epitope, combining the amino acids into a linear peptide, performing directed permutations, synthesizing the DSP using solid phase chemistry, preparing the DSP as a pharmaceutically acceptable salt, introducing the DSP into a host, harvesting primary tissue containing antibody producing cells from the host after one week, alternatively harvesting primary tissue containing antibody producing cells from the host after a time greater one week, determining the activity of the generated antibodies, selecting the desired activity, and utilizing the antibody as a reagent or alternatively as a therapeutic.

Briefly, as the means to generate antibodies, antibodies of interest are identified using means known to one skilled in the art, for example, phage display library screening or B-cell proliferation screening. The antigen used is a novel composition comprising a mixture of peptides that are related to a target epitope. A method of the instant invention uses a sequence of a known peptide epitope as a starting point. The amino acids that make up the epitope are sequentially modified via the introduction of different, related amino acids defined by a set of rules. The result is a mixture of related peptides useful in and of itself as a therapeutic, which is described herein as a composition comprising “directed-sequence polymers” or “DSP”. Such composition is referred to as a “DSP composition.” The method of synthesizing a DSP composition utilizes and maintains the natural order of amino acid residues of a defined peptide sequence of a specified length. Each amino acid position is subjected to change based on a defined set of rules. In a preferred embodiment the amino acids is substituted according to the methods seen in Table X of Kosiol et al., J. Theoretical Biol., 2004, 228:97-106). Alternatively, amino acids can be changed in accordance with the exemplary substitutions described in PCT/US2004/032598, page 10-11. Alternatively, amino acids can be changed in accordance with the differences in amino acids in the source epitope. For the solid phase synthesis procedure of the instant invention, the mixture of amino acids for a given position in the peptide is defined by a ratio one to another. Prior to starting the synthesis, such ratio is determined for each position along the peptide. The resulting directed order peptide mixture comprises a multiplicity of related peptide sequences.

The length of a DSP can be one of the original defined sequence peptide or 30 lengths of the original defined sequence peptide. The length of the combined sequence can be between 25 and 300 amino acids.

The percentage of alanine as compared to all of the other amino acids in the DSP combined will always be greater than 10%, and will not exceed 90%. Preferably, the alanine percentage is between 20% and 80%. More preferably the percentage of alanine is between 40% and 75%. The complexity of the mixture is greater than 5×10² different peptides. Preferably the complexity of the mixture is greater than 1×10¹⁰ different peptides. More preferably the complexity of the mixture is greater than 1×10¹⁵ different peptides.

In some embodiments, the DSP is derived from cancer specific or cancer-enhanced proteins and epitopes. In other embodiments, the DSP is derived from autoimmune-related proteins and epitopes. In further embodiments, the DSP is derived from infectious disease related epitopes. Examples of proteins from which the DSP derive include G-protein coupled receptors (GPCR), inflammatory related proteins, allergic related proteins, interleukins and their receptors, chemokines and their receptors, chapperones and their receptors. In other embodiments, the DSP is derived from CD20, vascular endothelial growth factor (VEGF), CD52, epidermal growth factor receptor (EGFR+), CD33, HER2; non-oncology related proteins, e.g. TNF alpha, CD25 or immunoglobulin E, for immunosuppression, CD11a, alpha4-beta1 integrin; infectious disease related beta chemokine receptor CCR5, RSVgpP. In other embodiments, the DSP is derived from empirically derived peptide sequences, such as through screening of library created by combinatorial chemistry.

In still further embodiments, the DSP is taken from the group proteins comprising: a protein known only as containing a domain having a primary, secondary tertiary or quaternary structural attribute, such as beta pleated sheet or alpha helicies, a protein known only as containing a domain having a certain activity, such as serotonin binding, a protein known only as having a known origin, a protein known only as belonging to a specific cellular compartment such as the nucleus or cytoplasm, a protein known only as having a cellular function, such as a cellular process producing a specific protein of interest, a protein known only as having an antioxidant activity or a metabolic activity, or a biosynthesis activity, or a catabolic activity, or a kinase activity, or a transferase activity, or a lyase activity, or a ligase activity, or a signal transduction activity or a binding activity, or a motility activity, or a membrane fusion activity, or a cellular communication activity, or a biological process regulation activity, response to stimulus activity, a cellular death related activity, a T cell activation related activity, a B cell activation related activity, an APC activation related activity, an inflammatory immune response related activity, an allergic response related activity, an infectious disease response related activity, a transporter activity, a channel activity, a secretion activity, a pathogenic activity, and a cytoskeleton organization activity.

An alternative embodiment of the instant invention encompasses methods for using DSP ligands in generating antibodies to proteins having humoral immunogenicity but not cellular immunogenicity. A further alternative embodiment of the instant invention encompasses methods for using DSP ligands in generating antibodies to proteins having cellular immunogenicity, but not humoral immunogenicity. A further embodiment of the instant invention encompasses using DSP ligands in generating antibodies against proteins with low levels of immunogenicity.

An alternative embodiment of the instant invention encompasses methods for using DSP ligands in generating antibodies to proteins having low levels of immunogenicity by combining a DSP ligand with a factor that increases humoral immunity, alternatively a factor that increases cellular immunity. An alternative embodiment of the instant invention encompasses methods for using DSP ligands in generating antibodies to proteins having low levels of immunogenicity by combining a DSP ligand with a factor taken from the group comprising: a factor that alters the foreignness of the protein, a factor that alters the size of the protein, a factor that alters the complexity of the protein, a factor that alters the chemical composition of the protein, and a factor that alters the antigen presentation of the protein.

### BRIEF DESCRIPTION OF THE DRAWINGS

FIG. 1 is a schematic for conceptual steps for generating Directed Sequence Polymers.

FIG. 2 shows the steps for preparing antibodies using Directed Sequence Polymers as a ligand.

FIG. 3 shows the preferred defined substitutive rules for directed expansion of epitope permeability.

FIG. 4 shows a generic rule structure and ranges of substitutions of DSP synthesis.

FIG. 5 shows an example of the application of the DSP Synthesis Rules using a mock-source peptide.

FIG. 6A-B shows examples of the application of the DSP Synthesis Rules using a CD20-derived peptide as a source peptide.

FIG. 7A-B shows an example of the application of the DSP Synthesis Rules using Gp100 (a.a. residues 154-162) as a source peptide.

FIG. 8A-B shows examples of the application of the DSP Synthesis Rules using an HLA-derived peptide and an HLA mimic-derived peptide as source peptides.

FIG. 9A-B shows an example of the application of the DSP Synthesis Rules using a hTRT-derived epitope peptide as a source peptide and applying an empirically determined substitution rule.

### DETAILED DESCRIPTION OF THE INVENTION

Drug discovery can be generalized into two major elements, lead generation and lead optimization. The development and exploitation of combinatorial chemistry (CC) has seen the divergence of the uses of rational design versus random generation on a very fundamental level. On one side we find the use of CC to assist a researcher in the rational design of molecules. An example of which can be seen in the discovery of structure/activity relationships (SAR) between two or more active molecules of therapeutic interest.

On the other side we find researchers using CC to define for them the design of new molecules discovered based on a specific activity. An example of which would be the generation of random libraries used in lead generation, whereby the lead is singled out and further optimized.

The level of expertise in the state of the art of combinatorial chemistry as applied to the synthesis of peptide libraries has risen, producing highly reliable and pure mixtures of peptides of great diversity. The use of these diverse peptide libraries has focused on lead generation and optimization. This strategy entails screening the vast numbers of individual peptide sequences in the library against a target of interest with the intention of defining a single, or limited set of peptides which demonstrate a particular activity. That single peptide, or the limited set of peptides, then become candidates which are modified to increase activity against the target.

The challenge for practitioners in this art has been to deconvolute, or accurately define the single or limited set of peptides that were responsible for the observed activity. The difficulties associated with deconvolution have spawned great efforts on the part of practitioners to create synthesis methods which inherently increase the resolution of individual peptides, as well as the identity of individual amino acids within peptides.

This knowledge has been applied to the process of selection of antibodies for pharmaceutical uses. For antibody selection, the library of antibodies may be phage display library, a library of humanized antibodies, or a population of B cells from a patient afflicted with a disease for which the antibody is screened.

Despite all the improvement in the technology, as powerful and clear cut the identification of a specific antibody from a combinatorial library may be, it often only serves as a starting point and identification of a lead antibody that is not itself therapeutically useful. The identified antibody may not block the activity of the target protein, or possibly exacerbate the very condition that the therapy aims to relieve. Such antibody is not directly therapeutically useful. However, one may create, by mutagenesis and protein engineering based on such antibody, antibodies that would have functionalities that are therapeutically useful.

Screening methods for antibodies are commonly designed to identify those antibodies that bind to a target epitope. It is important to select as a target an epitope that is relevant to the therapeutic usefulness of the identified antibodies. This consideration is particularly important in diseases where epitope spreading is seen. To increase the likelihood of identifying relevant antibodies, the target epitope may be manipulated.

Using a defined peptide or a set of peptides is advantageous over using a whole protein because of the ability to control and consistently produce uniform samples. In a native protein, natural phenomena such as the degree, the kind, and reproducibility of glycosylation, proper folding of the protein, and degradation and/or physiological activity of the protein must be considered. Purification and isolation from other cellular materials may sometimes pose a challenge.

Epitopes determined as related to a disease or a condition by various methodologies as further described below can be modified to expand the kinds of antibodies that would not be identified by using the original epitopes as the screening target for reasons such as attenuated binding to the original target (which antibody may yet be physiologically effective). In addition, to identify a collection of related but different antibodies, a series of such modified epitopes may be useful.

It has been observed for some time that in the course of development of multiple sclerosis, the reactive epitope does not stay constant. That is, the self recognition associated with the development of MS is a developmental process characterized by autoreactive diversity, plasticity, and instability, wherein the target epitope changes over time, typically from one epitope on a myelin proteolipid protein to one overlapping the amino acid residues but shifting by one or few amino acids to either side of the original epitope. The consequence of this phenomenon is that if an immunotherapeutic drug was targeted at the original epitope, over time, it becomes ineffective, not because of resistance to the mechanism of the drug, but simply because the target is no longer valid. J. Clin. Invest., 1997, 99:1682-1690. Thus, a collection of related antibodies may be effective in counteracting the series of undesired antibodies generated by the host in a serial manner.

It has previously been shown that mixtures of related peptides may be therapeutically more effective than a single peptide. Lustgarten et al., J. Immunol. 2006, 176: 1796-1805; Quandt et al., Molec. Immunol. 2003, 40: 1075-1087. The effectiveness of a peptide mixture as opposed to a single peptide is the likelihood of interaction with the broadening of the offending epitopes via the process of epitope spreading. (Immunol. Rev. 1998, 164:241) Therefore, to increase and maintain the effectiveness, these previous treatment modalities have been modified. For example, a therapeutic composition based on an altered peptide ligand (APL) method may include multiple peptides created from the original epitope by altering a small number of amino acid residues within the epitope sequence, in combination with the original epitope peptide, or other APLs. Fairchild et al., Curr. Topics Peptide & Protein Res. 6, 2004. Each APL would have a defined sequence, but the composition may be a mixture of APLs with more than one sequence. Using such mixture as an antigenic composition, a collection of related antibodies may be identified.

Another method that may identify a collection of related antibodies is to use random sequence copolymers as the epitope to screen for such antibodies. Random sequence copolymers are a collection of peptides having a defined amino acid composition but not defined sequences. A well-known example is COP-1, a mixture of peptides having an overall composition of Y, E, A, K, in a certain ratio, but for which the sequence of these amino acid residues are not prescribed. As a therapeutic agent, there have been a number of approaches to improve upon COP-1 by varying the amino acid contents and the ratios of the amino acids; however, the shortcoming of using RSP remains. For improved therapeutic RSP, see, for example, Strominger et al. (WO/2003/029276) and developed further by Rasmussen et al. (US 2006/0194725); WO/2005/032482; and WO/2005/074579.

The drawback of the these approaches is the undefined nature of what is effective in each motif, and quite possibly a large proportion of the peptides in the mixture may be inactive, lowering the concentration of useful epitopes. Additionally, these compounds are difficult to manufacture and to obtain consistency from lot to lot. Accordingly, the therapeutic usefulness of antibodies identified using these random copolymers are not strongly expected, making such screening less effective. The instant invention draws out the most useful properties of the previous methods of creating peptides useful for screening antibodies, yet removes the limitations of each.

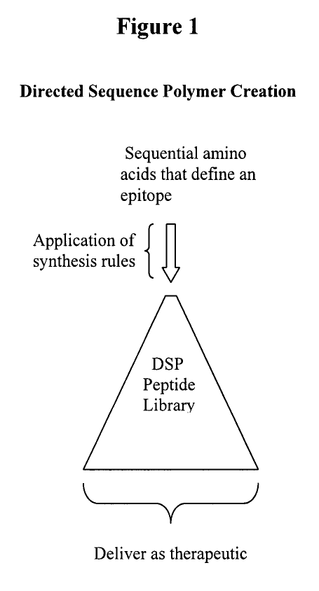

The instant invention relates to use of a “Directed Sequence Polymer” (DSP) to identify antibodies that are therapeutically effective. The approach is schematically represented in FIG. 1. A DSP is a peptide having a sequence derived from a base peptide sequence, which may be but not limited to a native epitope associated with an unwanted immune response. A DSP has one or more amino acid residue that differs from that of the base peptide sequence, the substitution of which is determined by a defined rule that is intended to preserve certain characteristics of the amino acid residue that is being replaced.

Antibodies induced by a DSP composition are expected to relate to those recognizing the base peptide but different. This difference is expected to be advantageous to identify antibodies that recognize epitopes that are not readily exposed, for example, epitopes that are transition conformations or epitopes that are half obscured in the native state. These epitopes, called “opaque” or “camouflaged” or “masked” epitopes, can nevertheless be accessed by conformationally different antibodies. Because of the high content of alanine, a small residue, DSP has more chances to mimic these potential epitopes.

Antibodies induced by a DSP composition may also be useful beyond the antibodies against the base peptide, because such antibodies are expected recognize and bind to the target in a way that differs from antibodies screened by their detectable binding to the base peptide and thus activate or inactivate a function of the target to a different degree or in a different manner than the antibodies against the base peptide.

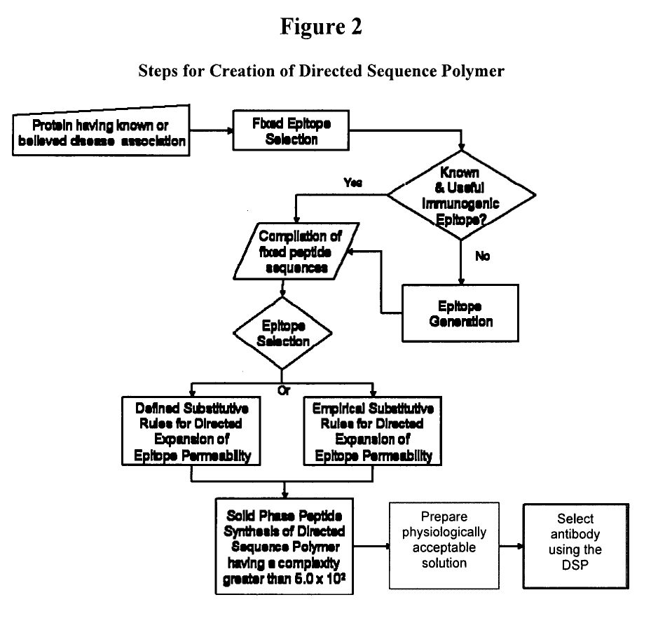

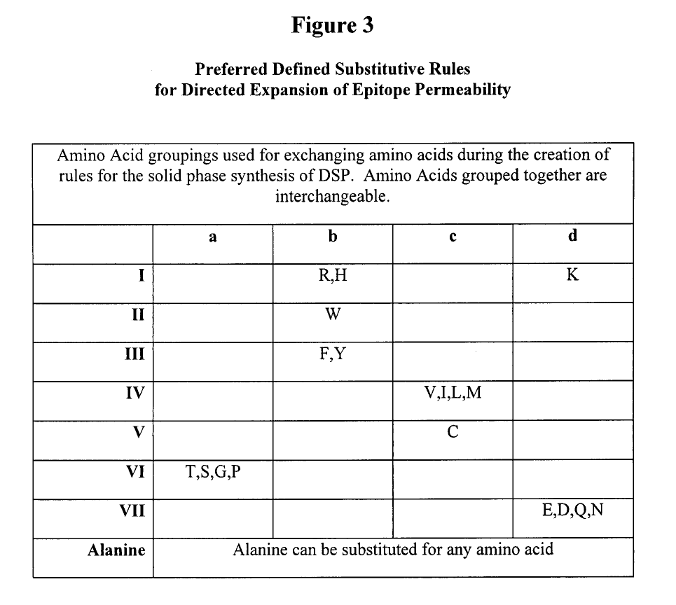

A DSP composition comprising multiple DSPs is synthesized by applying a set of synthesis rules that define the amino acid variations and the ratio of occurrence of introduction of such amino acid residues at any given position of the sequence to the base peptide sequence. Thus, a DSP is not synthesized as a single peptide, but is always synthesized as part of a composition comprising multiple related DSPs, the overall mixture of which is reproducible and consistent with the rules of synthesis that were applied. The schematic for the steps for creating a DSP composition, starting from the choice of a base peptide, is shown in FIG. 2.

### I. Base Peptide Sequences

To create a meaningful DSP composition, one first needs to define the base peptide sequence to derive the DSPs from. The base peptide sequences can be derived in many ways. A peptide sequence useful for this purpose is a peptide sequence that is known to be or thought to be a relevant target of antagonizing or agonizing that protein's activity. Some of these sequences have already been identified and have been used as targets for approved antibody therapeutic drugs. See, for example, Table 1 of Mascelli et al., J. Clin. Pharmacol. 2007, 47: 553-565 and Carter P. et al., AACR Education Book, AACR 96th Annual Meeting, Apr. 16-20, 2005, 147-154. Such antibodies, however, can still be improved by, for example, increasing the binding affinity to the target, or, preparing variations that would be effective for patients with genetic variations of the target, the original therapeutic antibodies do not react or react poorly with.

Cancer Related Polypeptides and Epitopes

These peptide sequences are, for example, cancer specific or cancer-enhanced proteins and epitopes, such cancer selected from the group consisting of leukemia, breast, skin, bone, prostate, liver, lung, brain, larynx, gallbladder, pancreas, rectum, parathyroid, thyroid, adrenal, neural, head and neck, colon, stomach, bronchi, kidneys, basal cell, carcinoma, squamous cell carcinoma, melanoma, metastatic skin carcinoma, osteo sarcoma, Ewing's sarcoma, veticulum cell carcinoma, myeloma, giant cell tumor, small-cell lung tumor, gallstones, islet cell tumor, primary brain tumor, lymphocytic, granulocytic, hairy-cell, adenoma, hyperplasia, medulliary carcinoma, pheochromocytoma, ovarian tumor, cervical dysplasia, in situ carcinoma, neuroblastoma, retinoblastoma, soft-tissue sarcoma, kaposi's sarcoma, osteogenic sarcoma.

More concretely, these proteins and epitopes are, e.g., G-protein coupled receptors (GPCR); CD20 (CALMIANSC (SEQ ID NO: 1), CWWEWTIGC (SEQ ID NO: 2), Binder et al. Blood 2006, 108: 1975-78), vascular endothelial growth factor (VEGF), CD52, epidermal growth factor receptor (EGFR+), CD33, HER2; non-oncology related proteins, e.g. TNF alpha; CD25 ((116)ERIYHFV(122) (SEQ ID NO: 4) and its structural analog CWYHYIWEC (SEQ ID NO: 5), Binder et al., Cancer Res. 2007, 67(8):3518-23) or immunoglobulin E for immunosuppression, CD11a, alpha4-beta1 integrin; infectious disease related beta chemokine receptor CCR5 or RSVgpP, and empirically derived peptide sequences, such as through screening of library created by a combinatory chemistry.

G Protein Coupled Receptors

G protein Coupled Receptors (GPCR), also known as seven transmembrane proteins (7-TM), are a large family of proteins that provide translation of extracellular stimuli into intracellular signals. The GPCR family of proteins is highly conserved amongst vertebrates and invertebrates. It is estimated that there is more than 800 GPCRs in the human genome (reviewed in Kroeze, W., J. Cell Science, 116:4867). An embodiment of the methods of the instant invention utilizes GPCR proteins as the basis for DSP, said GPCR(sequences readily available at http://www.expasy.org) taken from the group comprising:

5-hydroxytryptamine (serotonin) receptor 38; 5-hydroxytryptamine (serotonin) receptor 1A; 5-hydroxytryptamine (serotonin) receptor 1B; 5-hydroxytryptamine (serotonin) receptor 1D; 5-hydroxytryptamine (serotonin) receptor 1E; 5-hydroxytryptamine (serotonin) receptor 1F, 2A; 5-hydroxytryptamine (serotonin) receptor 2A; 5-hydroxytryptamine (serotonin) receptor 2B; 5-hydroxytryptamine (serotonin) receptor 2C; 5-hydroxytryptamine (serotonin) receptor 3A; 5-hydroxytryptamine (serotonin) receptor 4; 5-hydroxytryptamine (serotonin) receptor 5A; 5-hydroxytryptamine (serotonin) receptor 6; 5-hydroxytryptamine (serotonin) receptor 7 (adenylate cyclase-coupled); adenosine A 1 receptor; adenosine A2a receptor; adenosine A2b receptor; adenosine A3 receptor; adenylate cyclase activating polypeptide 1 (pituitary) receptor type I; adrenergic alpha-1A-receptor; adrenergic alpha-1B-receptor; adrenergic alpha-1D-receptor; adrenergic alpha-2A-receptor; adrenergic alpha-2B-receptor; adrenergic alpha-2C— receptor; adrenergic beta-1-receptor; adrenergic beta-2-receptor; adrenergic beta-3-receptor; adrenomedullin receptor; angiotensin II receptor type 1; angiotensin II receptor type 2; angiotensin II receptor-like 1; arginine vasopressin receptor 1A; arginine vasopressin receptor 1B; arginine vasopressin receptor 2 (nephrogenic diabetes insipidus); bombesin-like receptor 3; bradykinin receptor B1; bradykinin receptor B2; brain-specific angiogenesis inhibitor 1; brain-specific angiogenesis inhibitor 2; brain-specific angiogenesis inhibitor 3; Burkitt lymphoma receptor 1 GTP binding protein (chemokine (C-X-C motif) receptor 5); cadherin; calcitonin receptor; calcitonin receptor-like; calcium-sensing receptor (hypocalciuric hypercalcemia 1; cannabinoid receptor 1 (brain); cannabinoid receptor 2 (macrophage); CD97 molecule; chemokine (C-C motif) receptor 1; chemokine (C-C motif) receptor 2; chemokine (C-C motif) receptor 3; chemokine (C-C motif) receptor 4; chemokine (C-C motif) receptor 5; chemokine (C-C motif) receptor 6; chemokine (C-C motif) receptor 7; chemokine (C-C motif) receptor 8; chemokine (C-C motif) receptor 9; chemokine (C-C motif) receptor-like 1; chemokine (C-C motif) receptor-like 2; chemokine (C-X3-C motif) receptor 1; chemokine (C-X-C motif) receptor 4; chemokine (C-X-C motif) receptor 6; chemokine binding protein 2; chemokine orphan receptor 1; chemokine-like receptor 1; cholecystokinin A receptor; cholecystokinin B receptor; cholinergic receptor muscarinic 1; cholinergic receptor muscarinic 2; cholinergic receptor muscarinic 3; cholinergic receptor muscarinic 4; cholinergic receptor muscarinic 5; chromosome 7 open reading frame 9; coagulation factor II (thrombin) receptor; coagulation factor II (thrombin) receptor-like 1; coagulation factor II (thrombin) receptor-like 2; coagulation factor II (thrombin) receptor-like 3; complement component 3a receptor 1; complement component 5a receptor 1; corticotropin releasing hormone receptor 1; corticotropin releasing hormone receptor 2; cryptochrome 1 (photolyase-like); cysteinyl leukotriene receptor 1; cysteinyl leukotriene receptor 2; dopamine receptor D1; dopamine receptor D2; dopamine receptor D3; dopamine receptor D4; dopamine receptor D5; Duffy blood group chemokine receptor; EGF latrophilin and seven transmembrane domain containing 1; EGF LAG seven-pass G-type receptor 1 (flamingo homolog Drosophila); EGF LAG seven-pass G-type receptor 2 (flamingo homolog Drosophila); EGF LAG seven-pass G-type receptor 3 (flamingo homolog Drosophila) cadherin; egf-like module containing mucin-like hormone receptor-like 2; egf-like module containing mucin-like hormone receptor-like 3; egf-like module containing mucin-like hormone receptor-like 1; endothelial differentiation lysophosphatidic acid G-protein-coupled receptor 6; endothelial differentiation lysophosphatidic acid G-protein-coupled receptor 7; endothelial differentiation sphingolipid G-protein-coupled receptor 8; endothelial differentiation lysophosphatidic acid G-protein-coupled receptor 2; endothelial differentiation lysophosphatidic acid G-protein-coupled receptor; endothelial differentiation sphingolipid G-protein-coupled receptor 1; endothelial differentiation sphingolipid G-protein-coupled receptor 3; endothelial differentiation sphingolipid G-protein-coupled receptor 5; endothelin receptor type A; endothelin receptor type B cadherin; Epstein-Barr virus induced gene 2 (lymphocyte-specific G protein-coupled receptor); family with sequence similarity 62 (C2 domain containing) member A; follicle stimulating hormone receptor; formyl peptide receptor 1; formyl peptide receptor-like 1; formyl peptide receptor-like 2; frizzled homolog 1 (Drosophila); frizzled homolog 10 (Drosophila); frizzled homolog 2 (Drosophila); frizzled homolog 3 (Drosophila) (G protein-coupled receptor 68); frizzled homolog 4 (Drosophila); frizzled homolog 5 (Drosophila) (olfactory receptor family 2 subfamily H member 2); frizzled homolog 6 (Drosophila); frizzled homolog 7 (Drosophila); frizzled homolog 8 (Drosophila); frizzled homolog 9 (Drosophila); G protein-coupled bile acid receptor 1; G protein-coupled receptor family C group 5 member B; G protein-coupled receptor family C group 5 member C; G protein-coupled receptor family C group 5 member D; G protein-coupled receptor family C group 6 member A; G protein-coupled receptor 1 chemokine (C—C motif) receptor 10; G protein-coupled receptor 101; G protein-coupled receptor 103; G protein-coupled receptor 107; G protein-coupled receptor 108; G-protein-coupled receptor 109B; G protein-coupled receptor 110; G protein-coupled receptor 111; G protein-coupled receptor 112; G protein-coupled receptor 113; G protein-coupled receptor 114; G protein-coupled receptor 115; G protein-coupled receptor 116; G protein-coupled receptor 119; G protein-coupled receptor 12 (urotensin 2 receptor); G protein-coupled receptor 123; G protein-coupled receptor 124; G protein-coupled receptor 125; G protein-coupled receptor 126; G protein-coupled receptor 128; G protein-coupled receptor 132; G protein-coupled receptor 133; G protein-coupled receptor 135; G protein-coupled receptor 137; G protein-coupled receptor 139; G protein-coupled receptor 143; G protein-coupled receptor 146; G protein-coupled receptor 15; G protein-coupled receptor 150; G protein-coupled receptor 151; G protein-coupled receptor 152; G protein-coupled receptor 155; G protein-coupled receptor 156; G protein-coupled receptor 157; G protein-coupled receptor 158; G protein-coupled receptor 160; G protein-coupled receptor 161; G protein-coupled receptor 162; G protein-coupled receptor 17; G protein-coupled receptor 171; G protein-coupled receptor 172A; G protein-coupled receptor 172B; G protein-coupled receptor 173; G protein-coupled receptor 174; G protein-coupled receptor 175; G protein-coupled receptor 176; G protein-coupled receptor 18; G protein-coupled receptor 19; G protein-coupled receptor 20; G protein-coupled receptor 21; G protein-coupled receptor 22; G protein-coupled receptor 23 (melanin-concentrating hormone receptor 1); G protein-coupled receptor 25; G protein-coupled receptor 26; G protein-coupled receptor 27; G protein-coupled receptor 3; G protein-coupled receptor 30; G protein-coupled receptor 31; G protein-coupled receptor 32; G protein-coupled receptor 34; G protein-coupled receptor 35; G protein-coupled receptor 37 (endothelin receptor type B-like motilin receptor); G protein-coupled receptor 37 like 1; G protein-coupled receptor 39 (free fatty acid receptor 1, free fatty acid receptor 3); G protein-coupled receptor 4 (chemokine (C motif) receptor 1); G protein-coupled receptor 42 (free fatty acid receptor 2); G protein-coupled receptor 44; G protein-coupled receptor 45; G protein-coupled receptor 50; G protein-coupled receptor 52; G protein-coupled receptor 55; G protein-coupled receptor 56; G protein-coupled receptor 6 (neuropeptides B/W receptor 1, neuropeptides B/W receptor 2, chemokine (C-X-C motif) receptor 3, prolactin releasing hormone receptor); G protein-coupled receptor 61; G protein-coupled receptor 62; G protein-coupled receptor 63; G protein-coupled receptor 64; G protein-coupled receptor 65; G protein-coupled receptor 75; G protein-coupled receptor 77; G protein-coupled receptor 78; G protein-coupled receptor 81; G protein-coupled receptor 82; G protein-coupled receptor 83; G protein-coupled receptor 84; G protein-coupled receptor 85; G protein-coupled receptor 87; G protein-coupled receptor 88; G protein-coupled receptor 89A; G protein-coupled receptor 92; G protein-coupled receptor 97; G protein-coupled receptor 98; G protein-coupled receptor family C group 5 member A; galanin receptor 1; galanin receptor 2; galanin receptor 3; gamma transducing activity polypeptide 1; gamma-aminobutyric acid (GABA) B receptor 1; gamma-aminobutyric acid (GABA) B receptor 2; gastric inhibitory polypeptide-receptor; gastrin-releasing peptide receptor; GLI pathogenesis-related 1 like 1; glucagon receptor opsin 1 (cone pigments) medium-wave-sensitive (color blindness deutan); glucagon-like peptide 1 receptor; glucagon-like peptide 2 receptor; glutamate receptor metabotropic 1; glutamate receptor metabotropic 2; glutamate receptor metabotropic 3; glutamate receptor metabotropic 4; glutamate receptor metabotropic 5; glutamate receptor metabotropic 6; glutamate receptor metabotropic 7; glutamate receptor metabotropic 8; gonadotropin-releasing hormone (type 2) receptor 2; gonadotropin-releasing hormone receptor; growth hormone releasing hormone receptor; growth hormone secretagogue receptor; guanine nucleotide binding protein (G protein); HCRT; histamine receptor H1; histamine receptor H2; histamine receptor H3; histamine receptor H4; hypocretin (orexin) neuropeptide precursor; hypocretin (orexin) receptor 1; hypocretin (orexin) receptor 2; interleukin 8 receptor alpha; interleukin 8 receptor beta; KISS1 receptor; LanC lantibiotic synthetase component C-like 1 (bacterial); latrophilin 1; latrophilin 2; latrophilin 3; leucine-rich repeat-containing G protein-coupled receptor 4; leucine-rich repeat-containing G protein-coupled receptor 5; leucine-rich repeat-containing G protein-coupled receptor 6; leukotriene B4 receptor; leukotriene B4 receptor 2; luteinizing hormone/choriogonadotropin receptor; MAS1 oncogene; MAS1 oncogene-like; MAS-related GPR member F; MAS-related GPR member X1; MAS-related GPR member X2; MAS-related GPR member X3; MAS-related GPR member X4; melanin-concentrating hormone receptor 2; melanocortin 1 receptor (alpha melanocyte stimulating hormone receptor); melanocortin 2 receptor (adrenocorticotropic hormone); melanocortin 3 receptor; melanocortin 4 receptor; melanocortin 5 receptor; melatonin receptor 1A; melatonin receptor 1 B; natriuretic peptide receptor A/guanylate cyclase A (atrionatriuretic peptide receptor A); natriuretic peptide receptor B/guanylate cyclase B (atrionatriuretic peptide receptor B); natriuretic peptide receptor C/guanylate cyclase C (atrionatriuretic peptide receptor C); neuromedin B receptor; neuromedin U receptor 1; neuromedin U receptor 2; neuropeptide FF receptor 1; neuropeptide FF receptor 2; neuropeptide Y; neuropeptide Y receptor Y1; neuropeptide Y receptor Y2; neuropeptide Y receptor Y5; neurotensin receptor 1 (high affinity); neurotensin receptor 2; olfactory receptor family 1 subfamily A member 2; olfactory receptor family 1 subfamily E member 1; olfactory receptor family 1 subfamily E member 2; olfactory receptor family 1 subfamily G member 1; olfactory receptor family 1 subfamily D member 4; olfactory receptor family 1 subfamily J member 4; olfactory receptor family 10 subfamily A member 4; olfactory receptor family 10 subfamily A member 5; olfactory receptor family 10 subfamily AD member 1; olfactory receptor family 10 subfamily H member 1; olfactory receptor family 10 subfamily H member 2; olfactory receptor family 10 subfamily H member 3; olfactory receptor family 10 subfamily J member 1; olfactory receptor family 11 subfamily A member 1; olfactory receptor family 12 subfamily D member 2; olfactory receptor family 12 subfamily D member 3; olfactory receptor family 2 subfamily A member 4; olfactory receptor family 2 subfamily B member 11; olfactory receptor family 2 subfamily B member 2; olfactory receptor family 2 subfamily C member 3; olfactory receptor family 2 subfamily D member 2; olfactory receptor family 2 subfamily F member 1; olfactory receptor family 2 subfamily H member 1; olfactory receptor family 2 subfamily J member 2; olfactory receptor family 2 subfamily L member 13; olfactory receptor family 2 subfamily L member 3; olfactory receptor family 2 subfamily M member 4; olfactory receptor family 2 subfamily S member 2; olfactory receptor family 2 subfamily W member 1; olfactory receptor family 4 subfamily C member 11; olfactory receptor family 4 subfamily C member 3; olfactory receptor family 4 subfamily C member 6; olfactory receptor family 4 subfamily D member 1; olfactory receptor family 4 subfamily D member 2; olfactory receptor family 4 subfamily N member 4; olfactory receptor family 5 subfamily I member 1; olfactory receptor family 5 subfamily L member 2; olfactory receptor family 5 subfamily P member 2; olfactory receptor family 5 subfamily P member 3; olfactory receptor family 5 subfamily V member 1; olfactory receptor family 51 subfamily B member 2; olfactory receptor family 51 subfamily B member 4; olfactory receptor family 51 subfamily E member 1; olfactory receptor family 51 subfamily E member 2; olfactory receptor family 52 subfamily A member 1; olfactory receptor family 52 subfamily B member 4; olfactory receptor family 52 subfamily H member 1; olfactory receptor family 56 subfamily B member 4; olfactory receptor family 6 subfamily A member 2; olfactory receptor family 6 subfamily B member 3; olfactory receptor family 6 subfamily W member 1 pseudogene; olfactory receptor family 7 subfamily A member 17; olfactory receptor family 7 subfamily A member 5; olfactory receptor family 7 subfamily C member 2; olfactory receptor family 7 subfamily D member 2; olfactory receptor family 7 subfamily D member 4; olfactory receptor family 7 subfamily E member 5 pseudogene; olfactory receptor family 7 subfamily E member 91 pseudogene; olfactory receptor family 8 subfamily B member 8; olfactory receptor family 8 subfamily D member 1; olfactory receptor family 8 subfamily D member 2; olfactory receptor family 8 subfamily G member 2; olfactory receptor family 8 subfamily G member 5; olfactory receptor family 8 subfamily U member 1; olfactory receptor family 1 subfamily A member 1; olfactory receptor family 1 subfamily D member 2; olfactory receptor family 1 subfamily F member 1; olfactory receptor family 2 subfamily C member 1; olfactory receptor family 3 subfamily A member 1; olfactory receptor family 3 subfamily A member 2; olfactory receptor family 3 subfamily A member 3; olfactory receptor family 6 subfamily B member 2; opiate receptor-like 1; opioid receptor delta 1; opioid receptor kappa 1; opioid receptor mu 1; opsin 1 (cone pigments) long-wave-sensitive (color blindness protan); opsin 1 (cone pigments) short-wave-sensitive (color blindness, triton); opsin 3 (encephalopsin panopsin); opsin 4 (melanopsin); opsin 5; oxoeicosanoid (OXE) receptor 1; oxoglutarate (alpha-ketoglutarate) receptor 1; oxytocin receptor; pancreatic polypeptide receptor 1; parathyroid hormone receptor 1; parathyroid hormone receptor 2; platelet-activating factor receptor; progestin and adipoQ receptor family member VII; progestin and adipoQ receptor family member VIII; prokineticin receptor 1; prokineticin receptor 2; proline-rich protein PRP2; prostaglandin D2 receptor (DP); prostaglandin E receptor 1 (subtype EP1) 42 kDa; prostaglandin E receptor 2 (subtype EP2) 53 kDa; prostaglandin E receptor 3 (subtype EP3); prostaglandin E receptor 4 (subtype EP4); prostaglandin F receptor (FP); prostaglandin 12 (prostacyclin) receptor (IP); purinergic receptor P2Y G-protein coupled 10; purinergic receptor P2Y G-protein coupled 12; purinergic receptor P2Y G-protein coupled 13; purinergic receptor P2Y G-protein coupled 14; purinergic receptor P2Y G-protein coupled 5; purinergic receptor P2Y, G-protein coupled 1; purinergic receptor P2Y, G-protein coupled 11; purinergic receptor P2Y, G-protein coupled 2; pyrimidinergic receptor P2Y, G-protein coupled 4; pyrimidinergic receptor P2Y, G-protein coupled 6; relaxin/insulin-like family peptide receptor 1; relaxin/insulin-like family peptide receptor 2; relaxin/insulin-like family peptide receptor 3; retinal degeneration slow retinal G protein coupled receptor; retinal outer segment membrane protein 1; retinal pigment epithelium-derived rhodopsin homolog; rhodopsin (opsin 2; rod pigment) (retinitis pigmentosa 4 autosomal dominant); secretin receptor; serum amyloid A2; severe neonatal hyperparathyroidism); signal sequence receptor alpha (translocon-associated protein alpha); signal sequence receptor beta (translocon-associated protein beta); smoothened homolog (Drosophila); somatostatin receptor 1; somatostatin receptor 2; somatostatin receptor 3; somatostatin receptor 4; somatostatin receptor 5; succinate receptor 1; surface; tachykinin receptor 1; tachykinin receptor 2; tachykinin receptor 3; taste receptor type 1 member 1; taste receptor type 1 member 2; taste receptor type 2 member 1; taste receptor type 2 member 16; taste receptor type 2 member 3; taste receptor type 2 member 4; taste receptor type 2 member 9; taste receptor type 2 member 38; taste receptor type 2 member 5; thromboxane A2 receptor G protein-coupled receptor 137B; thyroid stimulating hormone receptor; thyrotropin-releasing hormone receptor; trace amine associated receptor 1; trace amine associated receptor 2; trace amine associated receptor 3; trace amine associated receptor 5; trace amine associated receptor 8; trace amine associated receptor 9; transmembrane 7 superfamily member 3; transmembrane protein 11; transmembrane protein 86B; vasoactive intestinal peptide receptor 1; vasoactive intestinal peptide receptor 2; vomeronasal 1 receptor 1; xenotropic and polytropic retrovirus receptor;

### Infectious Disease Agents

In other embodiments, such base peptide sequence is an epitope relevant to the pathology of a viral infectious disease selected from the group consisting of AIDS, AIDS Related Complex, Chickenpox (Varicella), Common cold, Cytomegalovirus Infection, Colorado tick fever, Dengue fever, Ebola haemorrhagic fever, Hand, foot and mouth disease, Hepatitis, Herpes simplex, Herpes zoster, HPV, Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease, and Yellow fever.

In other embodiments, such base peptide sequence is an epitope relevant to the pathology of a bacterial infectious disease selected from the group consisting of Anthrax, Bacterial Meningitis, Botulism, Brucellosis, Campylobacteriosis, Cat Scratch Disease, Cholera, Diphtheria, Gonorrhea, Impetigo, Legionellosis, Leprosy (Hansen's Disease), Leptospirosis, Listeriosis, Lyme disease, Melioidosis, MRSA infection, Nocardiosis, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Typhus (including epidemic typhus), and Urinary Tract Infections.

In other embodiments, such base peptide sequence is an epitope relevant to the pathology of a parasitic infectious disease selected from the group consisting of Amoebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amoebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Plasmodium, Scabies, Schistosomiasis, Taeniasis, Toxocariasis, Toxoplasmosis, Trichinellosis, Trichinosis, Trichuriasis, Trichomoniasis, and Trypanosomiasis (including African trypanosomiasis).

Some examples of epitope sequences useful for antibody production and as vaccine are listed in the table below:

| | | Source/ | | | SEQ |
|---|---|---|---|---|---|
| | | Original | Residue | | ID |
| Relevance | Peptide Sequence | Protein | Number | Ref | NO: |
| Generalized | KFGADARALMLQGVDLLADA | human HSP60 | 31-50 | 1 | 6 |
| Immune | | | | | |
| Activation | | | | | |
| | LKVGLQVVAVKAPGF | human HSP60 | 291-305 | 2 | 7 |
| | GGAVFGEEGLTLNLE | human HSP60 | 321-335 | 2 | 8 |
| | TLNLEDVQPHDLGKV | human HSP60 | 331-345 | 2 | 9 |
| | VGAATEIEMKEKKDR | human HSP60 | 381-395 | 2 | 10 |
| | VGGTSDVEVNEKKDR | human HSP60 | 406-420 | 2 | 11 |
| | IVLGGGCALLRCIPA | human HSP60 | 436-450 | 2 | 12 |
| | VLGGGVALLRVIPALDSLTPANED | human hsp60 | 437-460 | 3 | 13 |
| | GCALLRCIPALDSLT | human HSP60 | 441-455 | 2 | 14 |
| | RCIPALDSLTPANED | human HSP60 | 446-460 | 2 | 15 |
| | EIIKRTLKIPAMTIA | human HSP60 | 446-480 | 2 | 16 |
| | VEKIMQSSSEVGYDA | human HSP60 | 491-505 | 2 | 17 |
| | MAGDFVNMVEKGIID | human HSP60 | 506-520 | 2 | 18 |
| | VNMVEKGIIDPTKVV | human HSP60 | 511-525 | 2 | 19 |
| | VAVTMGPKGRTVIIE | human HSP60 | 51-65 | 2 | 20 |
| | KGIIDPTKVVRTALL | human HSP60 | 516-530 | 2 | 21 |
| | PTKVVRTALLDAAGV | human HSP60 | 521-535 | 2 | 22 |
| | ASLLTTAEVVVTEIP | human HSP60 | 536-550 | 2 | 23 |
| | GETRKVKAH | HLA-A2 | 62-70 | 4 | 24 |
| | RKVKAHSQTHRVDLG | HLA-A2 | 65-79 | 4 | 25 |
| | RVDLGTLRGYYNQSE | HLA-A2 | 75-89 | 4 | 26 |
| | DGRLLRGHDQYAYDG | HLA-B7 | 106-120 | 4 | 27 |
| | GPEYWDRNTQIYKA | HLA-B7 | 56-69 | 4 | 28 |
| | WDRNTQIYKAQAQTDR | HLA-B7 | 60-75 | 4 | 29 |
| | RNTQIYKAQ | HLA-B7 | 62-70 | 4 | 30 |
| | RESLRNLRGYYNQSE | HLA-B7 | 75-89 | 4 | 31 |
| | GSHTLQSMYGCDVGP | HLA-B7 | 91-105 | 4 | 32 |
| | LNEDLRSWTAAD | HLA-B7 | 150-161 | 5 | 33 |
| | LNEDLRSWTAABTAA | HLA-B7 | 150-164 | 5 | 34 |
| | DKGQVLNIQ | HLA-DQ2 | 133-142 | 6 | 35 |
| | LEDKGQVLNIQMRR | HLA-DQ2 | 131-144 | 6 | 36 |
| | AFKGSIFVVFDSIE | HLA-DQ2 | 149-162 | 6 | 37 |
| | ESAKKFVET | HLA-DQ2 | 162-170 | 6 | 38 |
| | IESAKKFVETPGQK | HLA-DQ2 | 161-174 | 6 | 39 |
| | AKDANNGNLQLR | HLA-DQ2 | 286-297 | 6 | 40 |
| | EALKKIIED | HLA-DQ2 | 311-324 | 6 | 41 |
| | EQIKLDEGW | HLA-DQ2 | 36-47 | 6 | 42 |
| | LKEQIKLDEGWV | HLA-DQ2 | 36-47 | 6 | 43 |
| | AELMEISED | HLA-DQ2 | 75-87 | 6 | 44 |
| | SKAELMEISEDKT | HLA-DQ2 | 75-87 | 6 | 45 |
| | KGSIFVVFD | HLA-DQ2, DQ7 | 149-162 | 6 | 46 |
| | AKDANNGNLQLRNK | HLA-DQ2, DQ7 | 286-299 | 6 | 47 |
| | DANNGNLQL | HLA-DQ2, DQ7 | 288-299 | 6 | 48 |
| | IVEALSKSKAEL | HLA-DQ2, DQ7 | 66-80 | 6 | 49 |
| | AFKGSIFWFDSI | HLA-DQ7 | 149-161 | 6 | 50 |
| | GSIFVVFDSIESAK | HLA-DQ7 | 152-165 | 6 | 51 |
| | IFVVFDSIESAKKF | HLA-DQ7 | 154-167 | 6 | 52 |
| | VVFDSIESA | HLA-DQ7 | 154-167 | 6 | 53 |
| | ELMEISEDKTKIR | HLA-DQ7 | 78-90 | 6 | 54 |
| | EALYLVCGE | HLA-DQ8 | 35-47 | 6 | 55 |
| Cancer | KTWGQYWQV | Gp100 | 154-162 | 7 | 56 |
| | KTWGQYWQVL | Gp100 | 154-164 | 17 | 57 |
| | ITDQVPFSV | Gp100 | 209-217 | 7; 8; 9 | 58 |
| | TITDQVPFSV | Gp100 | 208-217 | 17 | 59 |
| | LLDGTATLRL | Gp100 | | 17 | 60 |
| | VLYRYGSFSV | Gp100 | | 17 | 61 |
| | VLKRCLLHL | Gp100 | | 17 | 62 |
| | ALDGGNKHFL | Gp100 | | 17 | 63 |
| | VLPSPACQLV | Gp100 | | 17 | 64 |
| | YLEPGPVTA | Gp100 | 280-288 | 17 | 65 |
| | SLADTNSLAV | Gp100 | | 17 | 66 |
| | SVSVSQLRA | Gp100 | | 17 | 67 |
| | LNVSLADTN | Gp100 | | 17 | 68 |
| | SLYSFPEPEA | PRA | 100-108 | 10 | 69 |
| | SVYDFFVWL | TRP-2 | 180-188 | 11 | 70 |
| | ELAGIGILTV | MART-1 | 26-35 | 12 | 71 |
| | AAGIGILTV | MART-1 | | 17 | 72 |
| | EAAGIGILTV | MART-1 | | 17 | 73 |
| | AAGIGILTVI | MART-1 | | 17 | 74 |
| | KMVELVHFL | MAGE-2 | 112-120 | 13 | 75 |
| | RLFFYRKSV | HTRTp572 | 572-580 | 14 | 76 |
| Virus | ILARNLVPMV | HCMVpp65 | 491-500 | 15 | 77 |
| | ELEGVWQPA | HCMVpp65 | 526-534 | | 78 |
| | RIFAELEGV | HCMVpp65 | 522-530 | | 79 |
| | NLVPMVATV | HCMVpp65 | 495-503 | | 80 |
| | RIQRGPGRAFVTIGK | HIV-gp120 | V3 loop | 16 | 81 |

### Empirically Derived Base Peptide Sequences

As described in the above sections, peptide sequences with some significance to a disease state or an adverse reaction may be identified through experimental investigation of a relevant epitope. These sequences may include non-naturally occurring peptide sequences that proved to be useful in treating a disease or a condition, an example found in the international patent application publication WO 2006/031727, U.S. Pat. No. 6,930,168 and the related scientific publication Stern et al., Proc. Nat. Acad. Sci. USA, 2005, 102:1620-25.

Further, epitopes are empirically determined by identifying candidate sequences by positional scanning of synthetic combinatorial peptide libraries (see, for example, D. Wilson et al., above; R. Houghten et al., above; Hernandez et al., Eur J. Immunol., 2004, 34:2331-41), or by making overlapping peptide sequences of the entire protein of interest, and testing those peptides for immune reactivity (using, for example, any readout assay useful for such purposes, described in Current Protocols in Immunology Edited by John E Coligan, Ada M Kruisbeek, David H Margulies, Ethan M Shevach, Warren Strober NIH, John Wiley & Sons) in an in vitro or in vivo assay system appropriate for the disease and species the epitope is sought for. For example, for the design of a multiple sclerosis drug, an example of an appropriate system uses cells that derive from human subjects with MS.

After identifying a candidate epitope, a probable set of additional related epitopes are generated using modeling and prediction algorithms described in readily available references, for example WO 2000/042559, align and analyze the predicted binding of these probable epitopes using available prediction methods described in, for example, WO 2005/103679, WO 2002/073193 and WO 99/45954. Selecting from the peptides having the highest predicted activity/binding, take 40% of the predicted sequences and acquire the percentage of any given amino acid at each position. Use those percentages to create the rules for amino acid incorporation into a DSP synthesis.

### Other Sources of Base Peptide Sequences

In addition to methodology and results described in the above sections, epitope sequences may be used as base peptide sequences, that are identified and included in the Immune Epitope Database, (available at http://www.immuneepitope.org/home.do, led by Alex Sette funded by the National Institute of Allergy and Infectious Diseases of the National Institute of Health, USA) or any sequences identified by processes performed and disclosed by commercial entities such as Mixtures Sciences of San Diego, or by Algonomics of Ghent Belgium.

### II. Rules of Synthesis for Directed Sequence Polymers

Steps in the creation of a DSP sequentially encompass the following:

(a) Identify a protein having known or believed association with a pathology.

(b) Select from within the protein a peptide or peptides, each having a fixed sequence, that are associated with the pathology and immunologically relevant. If no peptides have been described, then peptides useful in the treatment of the pathology of interest are created. One exemplary method is to create a library of peptides that collectively span the entire length of the protein of interest. This may be done by, for example, partial endopeptidase digestion or by peptide synthesis. The library is screened for immunologically relevant peptides using appropriate detection methods such as binding affinity determination using antibodies detected in the sera of patients with the target pathology. The peptides may be further examined for immunogenicity useful for the treatment of the pathology in an in vitro or in vivo experimental system.

(c) the amino acid substitutions are decided based on either of two sets of rules, defined or empirical and are set forth below;

(d) Solid phase synthesis of DSP according to the rules is performed, and pharmaceutically acceptable formulation the DSP is delivered as a therapeutic.

The rules of synthesis for a composition comprising DSPs are outlined below. Briefly, a DSP may be envisioned as a polypeptide having a defined length that is either the same length as or multiples of the length of the base peptide sequence. For each residue position of the base peptide sequence, one or more substitute residue is defined. The rule of synthesis defines the ratio among the original base peptide residue for that position, the first substitute residue, the second substitute residue, the third substitute residue, and an alanine, to occupy any given residue position.

The substitute residues are defined according either: (1) to a rational comparison and finding of similarities of relevant characteristics of the original residue with those of the substitute residue or (2) to a comparison of reported experimental results on the relative activities of actual peptides having slight variations from the base sequence. The substitute residues defined in either of these two approaches are termed “conserved substitution” herein.

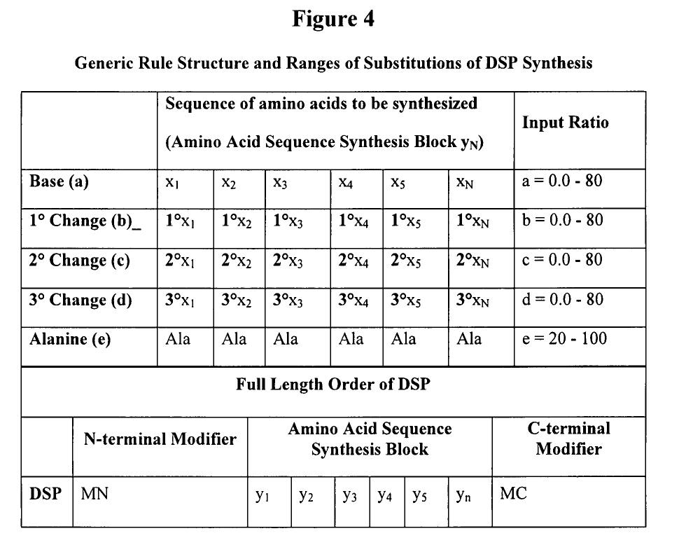

An example of a rational comparison and findings of similarity is the methods described by Kosiol et al., J. Theoretical Biol., 2004, 228:97-106. Amino acids are grouped together in a matrix, referred therein as PAM replacement matrix. FIG. 4 is a table showing the amino acid similarity and grouping, according to Kosiol, based on the characteristics of the residues such as size, charge, hydrophobicity, etc., as shown in Table X of the reference. In FIG. 4, amino acids grouped together are considered interchangeable, with high likelihood of retaining characteristics common among the group,

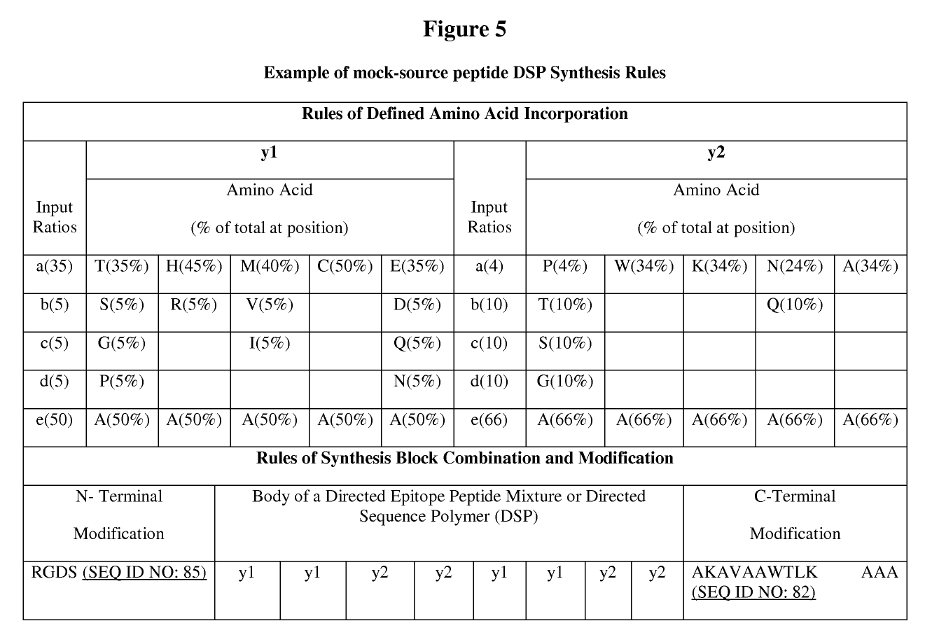

A comparison of experimental results showing the relative activities of peptides having slight variations from the base sequence can also be used as a basis for the rule for substitution. The sequences of the peptides responsible for observed changes are aligned and the type and percent presence of the new amino acid are noted. If there is more than one amino acid substitution at any given position of the peptide, the frequency of occurrence of an amino acid and the magnitude of activity change compared to the original sequence are taken into account to determine the order of prevalent substitution. Examples of the overall process leading up to the rule generation for DSP synthesis can be found using libraries (Molec. Immunol. 40:1047-1055; Molec. Immunol. 40:1063-74; J Autoimmunity 20:199-201; and J. Immunol. 163:6424-34), by making altered peptide ligands of overlapping peptides representing the entire protein of interest (Atkinson et al., J. Clin. Invest. 94:2125-29; Meini et al., J. Clin. Invest. 92:2633-43) or de novo (U.S. Pat. Nos. 7,058,515; 6,376,246; 6,368,861; 7,024,312; 6,376,246; 7,024,312; 6,961,664; 6,917,882). Briefly, a cellular material of interest is chosen as the assay system to rank the immunoreactivity of the peptides to be interrogated. Such an assay system can be either an in vitro or in vivo system, and can comprise adaptive or innate immune reactivity. Readouts for the assay system can be the up- or down-regulation of the status of the activation state of a protein, a change in the localization of a protein, the expression of the mRNA encoding for the protein, the relative concentration of a protein, changes in the generation of specific cell types, changes in cellular phenotype, changes in cellular activation, changes in cell number, changes in organ size or function, changes in animal behavior or phenotype. Once the assay or assays are performed the results are analyzed to determine the prevalence of any particular amino acid as a conserved substitution. If more than three residues in a given position within the peptide sequence are identified as generating a change in immunologic function, the top three residues first by frequency of representation in the interrogated peptides, and second by the magnitude of changes elicited. Once chosen, the relative amounts of the residues are defined. As depicted in FIG. 5, each cassette, “y”, has a set of amino acid ratios one to another that have a range of about 0-100 for the base (a), the primary change (h), the secondary change (c), and the tertiary change (d), whereas alanine (e) has a ratio of about 5-1000. The rules for the DSP synthesis continue with the combination of the cassettes in the order prescribed. The same block can be repeated either sequentially or separated by another block. On either side of the cassette sequence are N- and C-terminal modifiers. The number of cassettes is dictated by the requirements of the end length of the DSP which is required to be longer than 25 amino acids and shorter than 300 amino acids.

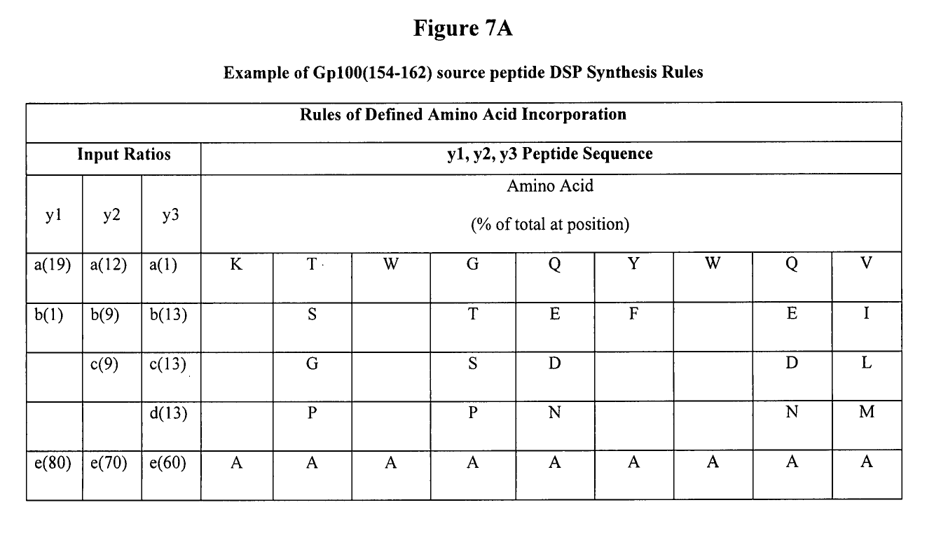

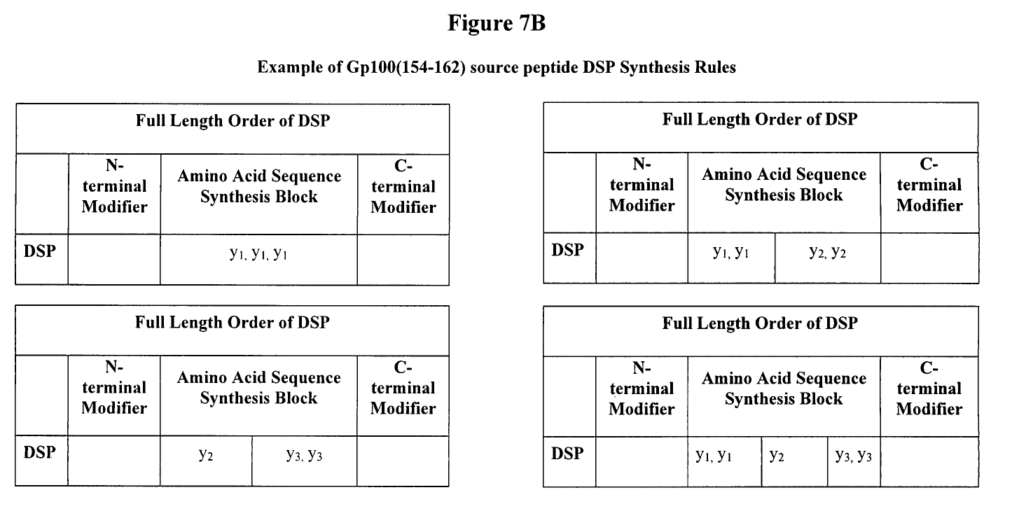

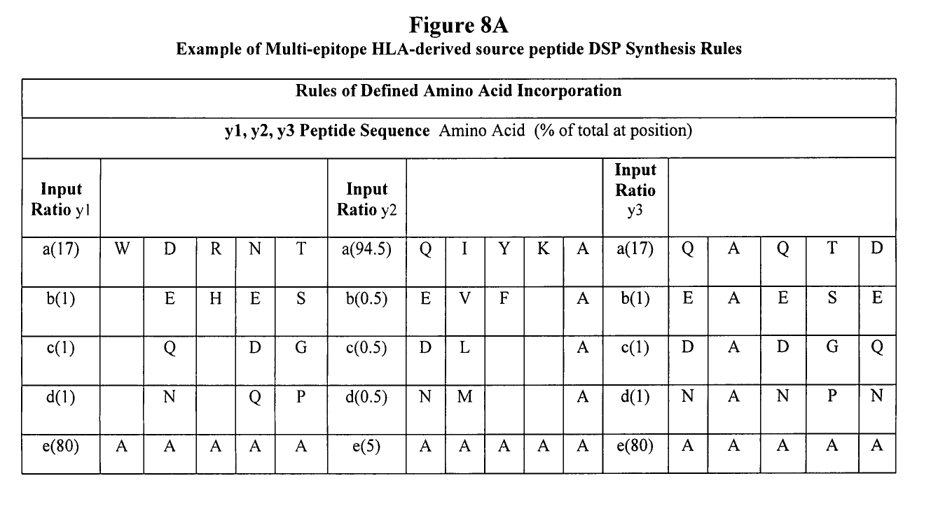

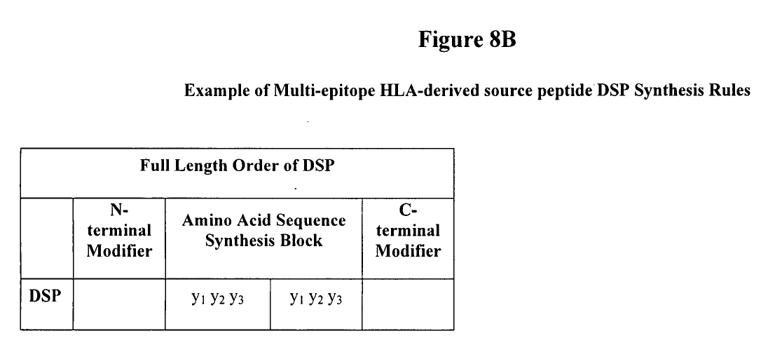

As described in FIG. 5, the instant invention envisions multiple epitopes to be defined as separate cassettes and synthesized sequentially. Cassette ratios within the same DSP may have different ratios of amino acids. Further, if there are less than three non-alanine amino acid substitutions, the percentage of the ‘missing’ substitution is added to the base sequence. Further, a cassette may be placed in any order with multiple appearances in the overall DSP synthesis. The N- and C-terminal Modifications reside prior to and after the entirety of the DSP cassettes respectively. As seen in FIG. 7A, a single base peptide sequence may have more than one ratio defined as a separate cassette in this example y1, y2, and y3. The individual cassettes can be placed in any order with multiple appearances in the overall DSP synthesis as seen in FIG. 7B. The synthesis rules seen in FIGS. 8A and 8B describe a DSP of the instant invention having portions of a single base peptide sequence with more than one ratio defined as a separate cassette.

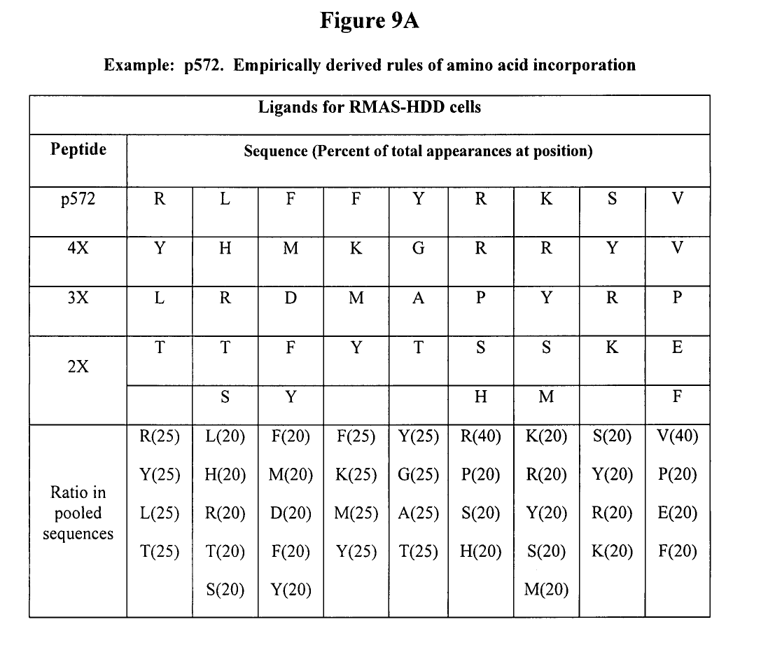

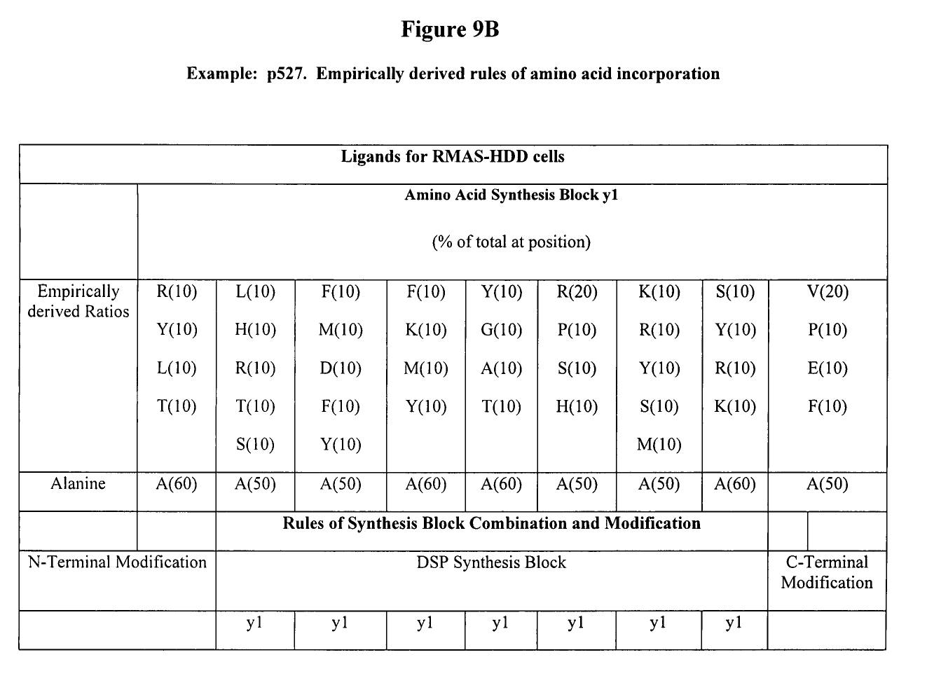

FIG. 9 demonstrates how the instant invention envisions empirically derived ratios of amino acids at a particular position. The example uses data derived from a T cell activation assay using diabetogenic T cells derived from transgenic NOD.BCD2.5 mice (J. Immunol. 166:908-17; J. Autoimmunity 20:199-201). The cells re interrogated with a combinatorial decamer library which resulted in a number of different peptides with inhibitory activity. The peptides with the highest activity were used to generate the amino acids at each position, as well as the ratio of different amino acids one to another.

A cassette may be repeated more than once. After a desired number of multiples of the cassette, if the desired length of the DSP is not yet reached, the DSP sequence is further defined by applying the same process, possibly using different ratio among the original, substitute, second substitute, and alanine residues.

In between the cassettes, amino acid sequences that assist epitope recognition may be added. For example, sequences known or likely to form beta-sheet structures, alpha helices, or bends may be introduced. See, for example, Mayo et al., Protein Sci., 1996, July;5(7):1301-15, for beta sheet motifs, Walshaw, J. et al., Biochem Soc Symp. 2001;(68):111-23 for coiled coil alpha helix motif, Karle, I L et al., Proc Natl Acad Sci USA 2000 Mar. 28; 97(7):3034-7 for helical and hairpin domains.

N or C-terminal DSP modifiers may be added to the synthesis rules. The purpose of such modifiers include but are not limited to enhancing binding to specific proteins as in the case of RDG-based amino acid sequences (U.S. Pat. Nos. 5,773,412; 5,770,565) used as targeting moieties, or peptides that are known to bind to a wide array of HLA-DR species, such as AKAVAAWTLK AAA (U.S. App. Pub. No. 2006/0018915) as a DR-targeting moiety. Such modifiers may include moieties which enhance complexation to delivery systems including sustained release delivery systems. Modifiers can be resorbable matrix constructs/synthesizable backbones such as PLGA. Modifiers can be protease resistant moieties such as D-amino acids.

Thus, for any given base peptide sequence, a set of synthesis rules is applied to yield a composition comprising reproducible, consistent mixture of DSPs.

### III. Peptide Synthesis Methods

Any known solid phase synthesis appropriate for peptide synthesis may be used to synthesize a composition comprising DSPs, for example as originally described by Merrifield (J. Am. Chem. Soc., 1963, 85:2149) and any variation thereof. More specifically, the synthesis is done in multiple steps by the Solid Phase Peptide Synthesis (SPPS) approach using Fmoc protected amino acids. SPPS is based on sequential addition of protected amino acid derivatives, with side chain protection where appropriate, to a polymeric support (bead). The base-labile Fmoc group is used for N-protection. After removing the protecting group (via piperidine hydrolysis) the next amino acid mixture is added using a coupling reagent (TBTU). After the final amino acid is coupled, the N-terminus is acetylated.

The resulting peptides (attached to the polymeric support through its C-terminus) are cleaved with TFA to yield the crude peptide. During this cleavage step, all of the side chains protecting groups are also cleaved. After precipitation with diisopropyl ether, the solid is filtered and dried. The resulting peptides are analyzed and stored at 2-8° C.

Additionally, any peptide synthesis method that allows synthesis incorporating more than one amino acid species at a controlled ratio in any given position of the peptide sequence is suitable for use with this invention. Further, as described below, DSPs may be peptidomimetics or include unnatural or modified amino acid, necessitating the adaptation to allow addition of such chemical species to the polymers synthesized up to that point.

The synthesis may include unnatural amino acids, or amino acid analogs. In some embodiments, the DSPs are comprised of naturally occurring and synthetic derivatives, for example, selenocysteine. Amino acids further include amino acid analogs. An amino acid “analog” is a chemically related form of the amino acid having a different configuration, for example, an isomer, or a D-configuration rather than an L-configuration, or an organic molecule with the approximate size and shape of the amino acid, or an amino acid with modification to the atoms that are involved in the peptide bond, so as to be protease resistant when polymerized in a polypeptide.

The DSPs for use in the present invention can be composed of L- or D-amino acids or mixtures thereof. As is known by those of skill in the art, L-amino acids occur in most natural proteins. However, D-amino acids are commercially available and can be substituted for some or all of the amino acids used to make DSPs of the present invention. The present invention contemplates DSPs containing both D- and L-amino acids, as well as DSPs consisting essentially of either L- or D-amino acids.

In certain embodiments, the DSPs of the present invention include such linear DSPs that are further modified by substituting or appending different chemical moieties. In one embodiment, such modification is at a residue location and in an amount sufficient to inhibit proteolytic degradation of the DSPs in a subject. For example, the amino acid modification may be the presence in the sequence of at least one proline residue; the residue is present in at least one of carboxy- and amino termini; further, the proline can be present within four residues of at least one of the carboxy- and amino-termini. Further, the amino acid modification may be the presence of a D-amino acid.

In certain embodiments, the subject DSPs is a peptidomimetic. Peptidomimetics are compounds based on, or derived from, peptides and proteins. The DSP peptidomimetics of the present invention typically can be obtained by structural modification of one or more native amino acid residues, e.g., using one or more unnatural amino acids, conformational restraints, isosteric replacement, and the like. The subject peptidomimetics constitute the continuum of structural space between peptides and non-peptide synthetic structures.

Such peptidomimetics can have such attributes as being non-hydrolyzableand may present similar but distinct conformation to identify antibodies that are related to but different from those easily identified using naturally occurring epitope peptides. For example, peptidomimetics may retain a conformation that the naturally occurring epitope peptide may not take as a peptide, but may be relevant as part of a whole protein or may be a transitional conformation. For illustrative purposes, peptide analogs of the present invention can be generated using, for example, benzodiazepines (e.g., see Freidinger et al. in “Peptides: Chemistry and Biology,” G. R. Marshall ed., ESCOM Publisher: Leiden, Netherlands, 1988), substituted gamma lactam rings (Garvey et al. in “Peptides: Chemistry and Biology,” G. R. Marshall ed., ESCOM Publisher: Leiden, Netherlands, 1988, p123), C-7 mimics (Huffman et al. in “Peptides: Chemistry and Biology,” G. R. Marshall ed., ESCOM Publisher: Leiden, Netherlands, 1988, p. 105), keto-methylene pseudopeptides (Ewenson et al. J. Med. Chem., 1986, 29:295; and Ewenson et al. in “Peptides: Structure and Function (Proceedings of the 9th American Peptide Symposium),” Pierce Chemical Co. Rockland, Ill., 1985), β-turn dipeptide cores (Nagai et al., Tetrahedron Lett., 1985 26:647; and Sato et al. J. Chem. Soc. Perkin Trans., 1986, 1:1231), β-aminoalcohols (Gordon et al. Biochem. Biophys. Res. Commun., 1985, 126:419; and Dann et al. Biochem. Biophys. Res. Commun., 1986, 134:71), diaminoketones (Natarajan et al. Biochem. Biophys. Res. Commun., 1984, 124:141), and methyleneamino-modified (Roark et al. in “Peptides: Chemistry and Biology,” G. R. Marshall ed., ESCOM Publisher: Leiden, Netherlands, 1988, p134). Also, see generally, Session III: Analytic and synthetic methods, in “Peptides: Chemistry and Biology,” G. R. Marshall ed., ESCOM Publisher: Leiden, Netherlands, 1988.

The molecular weight of a DSP composition can be adjusted during polypeptide synthesis or after the DSPs have been synthesized. To adjust the molecular weight during polypeptide synthesis, the synthetic conditions or the amounts of amino acids are adjusted so that synthesis stops when the polypeptide reaches the approximate length which is desired. After synthesis, polypeptides with the desired molecular weight can be obtained by any available size selection procedure, such as chromatography of the polypeptides on a molecular weight sizing column or gel, and collection of the molecular weight ranges desired. The present polypeptides can also be partially hydrolyzed to remove high molecular weight species, for example, by acid or enzymatic hydrolysis, and then purified to remove the acid or enzymes.

In one embodiment, the DSPs with a desired molecular weight may be prepared by a process which includes reacting a protected polypeptide with hydrobromic acid to form a trifluoroacetyl-polypeptide having the desired molecular weight profile. The reaction is performed for a time and at a temperature which is predetermined by one or more test reactions. During the test reaction, the time and temperature are varied and the molecular weight range of a given batch of test polypeptides is determined. The test conditions which provide the optimal molecular weight range for that batch of polypeptides are used for the batch. Thus, a trifluoroacetyl-polypeptide having the desired molecular weight profile can be produced by a process which includes reacting the protected polypeptide with hydrobromic acid for a time and at a temperature predetermined by test reaction. The trifluoroacetyl-polypeptide with the desired molecular weight profile is then further treated with an aqueous piperidine solution to form a low toxicity polypeptide having the desired molecular weight.

In one preferred embodiment, a test sample of protected polypeptide from a given batch is reacted with hydrobromic acid for about 10-50 hours at a temperature of about 20-28° C. The best conditions for that batch are determined by running several test reactions. For example, in one embodiment, the protected polypeptide is reacted with hydrobromic acid for about 17 hours at a temperature of about 26° C.

In certain embodiments, DSP is modified after synthesis. Such modification is useful, for instance, create DSP to direct the subsequent antibody response to features of the DSP that have application in either a research, diagnostic, or therapeutic context. Examples of post-synthesis modifications include but are not limited to sugars such as glycogen, alternative amino acids such as citrulline, phosphate moieties (pre-phosphorylated amino acids can also be added during synthesis), PEG additions of various lengths, biotin, fluorescent moieties, coupling to carrier proteins, alterations that form certain secondary structures such as a disulfide bridge, or modifications allowing for branching of the DSP though for example a lysine side chain. In one embodiment, the post-synthesis modification is performed using enzymes. In a further embodiment, the post-synthesis modification is performed manually using chemical complexation techniques well known in the prior art.

A further embodiment of the instant invention is the post-synthesis modification of the DSP by peptidylarginine deiminase. As an alpha-amino acid Citrulline has the formula C6H13N3O3. Citrulline has the following structure:

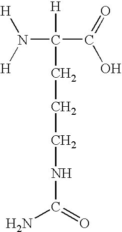

It is made from ornithine and carbamoyl phosphate in the urea cycle, as well as a by-product of arginine catalyzed by nitric oxide synthetase. Citrulline is not encoded for by DNA, but is added to proteins during post-translational modification events by peptidylarginine deiminases. Patient diagnosis with Rheumatoid Arthritis has been shown to correlate with immune responses to citrullinated proteins (Migliorini, P., Autoimmunity Reviews, 4:561-564). An embodiment of the instant invention is to create a citrullinated DSP as a lignad for antibodies to be used as a diagnostic for rheumatoid arthritis.

A further embodiment of the instant invention is the use of specific gylogenated forms of a DSP to create antibodies against such a form of a ligand. In one embodiment the ligand itself is an antibody. In one embodiment of the instant invention, the post-translational modification of a DSP is performed using glycogen synthase, or alternatively using chemical complexation techniques well known in the art.

### Definitions

The term “antibodies” means any immunoglobulin peptides, including but not limited to IgG, IgM, IgA, from any species or any fragments or any modified and/or engineered peptides derived from immunoglobulin, both single chain and multiple-chained, that (1) recognize a molecular structure comprising a target, (2) bind to the target by interacting with at least part of the molecular structure, and either (3) alter the physiological activity of the target or (4) alter the reaction of a host that harbors the target towards the target. Antibodies may be chimeric, for example as in humanized antibodies, and antibodies may be engineered by site directed mutagenesis of the CDR region of a naturally occurring peptide. Antibodies include not only full length and peptides that comprise the hypervariable region of a native immunoglobulin such as Fab and Fab′ fragments, but also short synthetic or engineered peptides that comprise the binding regions of naturally occurring antibodies, whether the binding regions comprise contiguous or noncontiguous peptide sequences. In the latter case, the synthetic or engineered peptides would comprise the peptide sequences of originally noncontiguous amino acid stretch as one contiguous sequence.

The term “associated with” means “coexistent with” or “in correlation with.” The term does not necessarily indicate causal relationship, though such relationship may exist.

The term “binding” refers to a direct association between two molecules, due to, for example, covalent, electrostatic, hydrophobic, ionic and/or hydrogen-bond interactions under physiological conditions, and including interactions such as salt bridges and water bridges.

The term “HLA molecule” means any class II major histocompatibility complex glycoproteins.

The term “immunomodulation” means the process of increasing or decreasing the immune system's ability to mount a response against a particular antigenic determinant through the T-cell receptor (“TCR”)'s recognition of complexes formed by major histocompatibility complex (“MHC”) and antigens.

The term “immunosuppression” means the depression of immune response and reactivity in recipients of organ or bone marrow allotransplants.

The term “MHC activity” refers to the ability of an MHC molecule to stimulate an immune response, e.g., by activating T cells. An inhibitor of MHC activity is capable of suppressing this activity, and thus inhibits the activation of T cells by MHC. In preferred embodiments, a subject inhibitor selectively inhibits activation by a particular class II MHC isotype or allotype. Such inhibitors may be capable of suppressing a particular undesirable MHC activity without interfering with all MHC activity in an organism, thereby selectively treating an unwanted immune response in an animal, such as a mammal, preferably a human, without compromising the animal's immune response in general.

The term “organ-specific protein” or “organ-specific antigen” means proteins that are expressed predominantly or exclusively by cells comprising a certain organ.

The term “patient” refers to an animal, preferably a mammal, including humans as well as livestock and other veterinary subjects.

The terms “peptide”, “polypeptide” and “protein” are used interchangeably herein. These terms refer to unmodified amino acid chains, and also include minor modifications, such as phosphorylations, glycosylations and lipid modifications. The terms “peptide” and “peptidomimetic” are not mutually exclusive and include substantial overlap.

A “peptidomimetic” includes any modified form of an amino acid chain, such as a phosphorylation, capping, fatty acid modification and including unnatural backbone and/or side chain structures. As described below, a peptidomimetic comprises the structural continuum between an amino acid chain and a non-peptide small molecule. Peptidomimetics generally retain a recognizable peptide-like polymer unit structure. Thus, a peptidomimetic may retain the function of binding to a HLA protein forming a complex which activates autoreactive T cells in a patient suffering from an autoimmune disease.

The term “amino acid residue” is known in the art. In general the abbreviations used herein for designating the amino acids and the protective groups are based on recommendations of the IUPAC-IUB Commission on Biochemical Nomenclature (see Biochemistry (1972) 11:1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid residue” further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Most of the amino acids used in the DSPs of the present invention may exist in particular geometric or stereoisomeric forms. In preferred embodiments, the amino acids used to form the subject DSPs are (L)-isomers, although (D)-isomers may be included in the DSPs such as at non-anchor positions or in the case of peptidomimetic versions of the DSPs.

“Prevent”, as used herein, means to delay or preclude the onset of, for example, one or more symptoms, of a disorder or condition.

“Treat”, as used herein, means at least lessening the severity or ameliorating the effects of, for example, one or more symptoms, of a disorder or condition.

“Treatment regimen” as used herein, encompasses therapeutic, palliative and prophylactic modalities of administration of one or more compositions comprising one or more DSP compositions. A particular treatment regimen may last for a period of time at a particular dosing pattern, which will vary depending upon the nature of the particular disease or disorder, its severity and the overall condition of the patient, and may extend from once daily, or more preferably once every 36 hours or 48 hours or longer, to once every month or several months.

The terms “structure-activity relationship” or “SAR” refer to the way in which altering the molecular structure of drugs alters their interaction with a receptor, enzyme, etc.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999; and PCR Protocols, ed. by Bartlett et al., Humana Press, 2003; PHARMACOLOGY A Pathophysiologic Approach Edited by Josehp T. DiPiro, Robert Talbert, Gary, Yee, Gary Matzke, Barbara Wells, and L. Michael Posey. 5th edition 2002 McGraw Hill; Pathologic Basis of Disease. Ramzi Cotran, Vinay Kumar, Tucker Collins. 6th Edition 1999. Saunders.

### Example 1

### Preparation of a DSP Composition from Fictitious Base Peptides

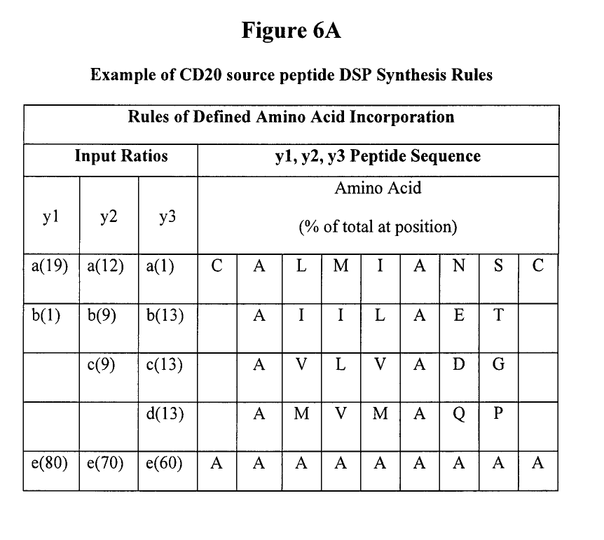

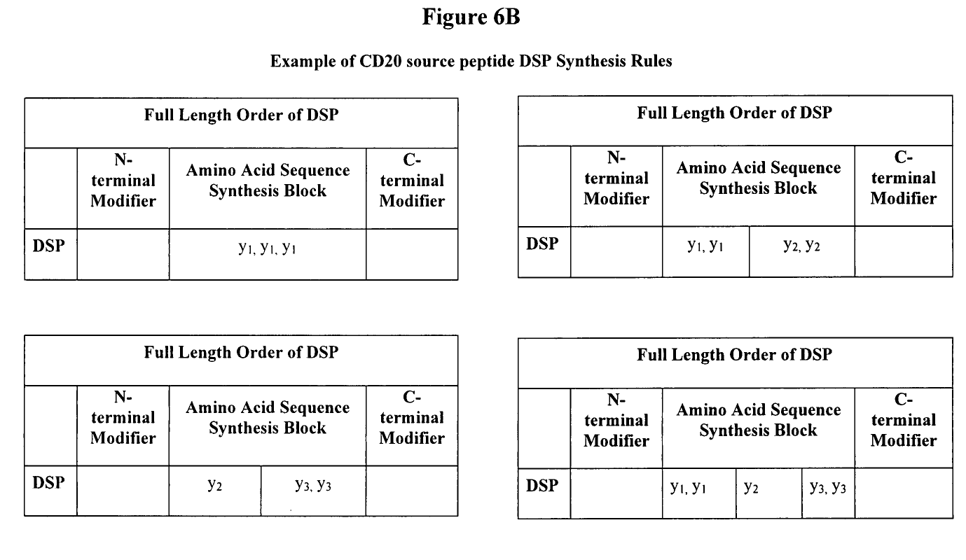

For ease of understanding, as an illustration, preparation of a DSP composition deriving from two fictitious peptide sequences, representing a known epitope, is described and shown in the table depicted in FIG. 6. In this illustration, the cassettes consist of five amino acids each, (x1, x2, x3, x4, x5=THMCE in y₁ and PWKNA in y₂).

THMCE is defined as having an input ratio of a=7, b=1, c=1, d=1, e=10. PWKNA is defined as having an input ratio of a=1, b=3, c=3, d=3, e=20. For synthesis, the identity of group of amino acids occupying each amino acid position for each peptide is determined using the preferred method of amino acid substitution described by Kosiol et al., J. Theoretical Biol. 228:97-106, 2004, as shown in FIG. 4 (or less preferably an equivalent means of systematically altering amino acids), and the overall ratio of amino acids that occupy each of such positions in the resulting collective DSP composition is given above. Each cassette, y₁ and y₂, will twice be repeated two times, generating an order of y₁ y₁ y₂ y₂ y₁ y₁ y₂ y₂. Nₙ are the number of times the sequence within the cassette is to be repeated, and in our fictitious example N=2. MN can be any type of modifying moiety. MN must be amenable to solid phase synthesis methods. For this fictitious example, a modifying moiety of amino acids that would target the DSP to a certain location within a subject is chosen, such as an RGD-based sequence motif on a particular integrin such as alphaVbeta3. In this example the C-terminal modifier will also be an RGD-based motif, but comprised of D-amino acids.

The DSP composition as described above is prepared using a solid phase peptide synthesis method as described elsewhere in this disclosure.

Using the DSP composition, a B cell library is screened by exposing the B cell library to the DSP composition and allowing self-selection of B cell lineage that bind a DSP and proliferate. The proliferating B cells are isolated and the CDR regions of the antibodies are sequenced to identify the antibodies to the DSP.

Alternatively, an immobilized DSP composition can be exposed to a phage display library expressing an array of antibodies. After incubating, unbound phages are washed away, and those bound to DSPs are isolated and sequenced.

### Example 2

### Preparation of a DSP Composition from Gp100 (a.a. Residues 154-162) As a Source Peptide

FIG. 7A-B shows an example of the application of the DSP Synthesis Rules using Gp100 (a.a. residues 154-162) as a source peptide. The methods and rules to define the identity of amino acids for each position of the resulting peptides are described above in Example 1. As with Example 1, the DSP composition is synthesized using a solid phase peptide synthesis method.

### Example 3

### Preparation of a DSP Composition from an HLA Peptide as a Source Peptide

FIG. 8A-B shows examples of the application of the DSP Synthesis Rules using an HLA-derived peptide and an HLA mimic-derived peptide as source peptides. The methods and rules to define the identity of amino acids for each position of the resulting peptides are described above in Example 1. As with Example 1, the DSP composition is synthesized using a solid phase peptide synthesis method.

### Example 4

### Preparation of a DSP Composition from an hTRT-Derived Epitope Peptide as a Source Peptide

FIG. 9A-B shows an example of the application of the DSP Synthesis Rules using a hTRT-derived epitope peptide as a source peptide and applying an empirically determined substitution rule. The methods and rules to define the identity of amino acids for each position of the resulting peptides are described above in Example 1. As with Example 1, the DSP composition is synthesized using a solid phase peptide synthesis method.

The following are a list of additional references the entirety of the content of each of which is incorporated by reference herein.
BERGTHORSDOTTIR, S. et al., J. Immunol. 2001, 166: 2228-2234.
BINDER, M. et al., Cancer Res. 2007, 67(8): 3518-3523.
BINDER, M. et al., Blood 2006, 108(6): 1975-1978.
BORUCHOV, A., J. Clin. Invest. 2005, 115(10): 2914-2923.
BUGLI, F. et al., J. Virol. 2001, 75(20): 9986-9990.
BREKKE, O. et al., Nature Reviews: Drug Discovery, 2003, 2: 52-62.
CARLO-STELLA, C., Cancer Res, 2006, 66(3): 1799-1801.
CARTER, P. et al., Educational Session: Therapeutic Antibodies in Cancer: Focus on Mechanism of Action, AACR 96th Annual Meeting, 2005, 147-154.
CARTON, G. et al., Blood, 2004, 104(9): 2635-2642.
CHEN, Z. et al., J. Immunol. 2000, 164:4522-4532.
CLACKSON, T. et al., Nature 1991, 352: 624-628.
DAL PORTO, J. et al., J. Immunol. 1998, 161:5373-5381.
FURIE, B. et al., J. Biol. Chem. 1978, 353(24): 8980-8967.
HAN, S. et al., Int'l Immunol. 2004, 16(4): 525-532.
HE, Y. et al., J. Immunol., 2002, 169: 594-605.
HOOGENBOON, H., Nature Biotechnol. 2005, 23(9): 1105-1116.
HOUGHTON, R. et al., 1991, 354:84-86.
HUST, M. et al., TRENDS in Biotechnol. 2004, 22(1): 8-14.
JACOB., J. et al., Nature 1991, 354: 389-392.
JENSEN-JAROLIM, E., Blood 2006, 108(6): 1794-1795.
KNAPPIK, A. et al., J. Mol. Biol. 2000, 296: 57-86.
KIRSCH, M. et al., J. Immunol. Methods 2005, 301: 173-185.
KONTHUR, Z. et al., Gene 2005, 364:19-29.
LIBERTI, P. et al., Biochemistry, 1971, 10(9):1632.
MASCELLI, M. et al., J. Clin. Pharmacol., 2007, 47:553-565.
MCKEAN, D. et al., Proc. Natl. Acad. Sci, USA 1984, 81:3180-3184.
OSBOURN, J. et al., Drug Discovery Today 2003, 8(18): 845-851.
NEMAZEE, D. et al., J. Exp. Med. 2000, 191(11): 1813-1817.
PERSSON, M. et al., Proc. Natl. Acad. Sci. USA, 1991, 88: 2432-2436.
PAUS, D. et al., J. Exp. Med. 2006, 203(4): 1081-1091.
PUFFINBARGER, N. et al., Molec. Pharmacol. 1995, 47: 1126-1132.
ZHANG, N. et al., Clin. Cancer Res. 2005, 11(16): 5971-5980.

The following references are exemplary sources of epitopes useful as base peptide sequences. Numbers to the left are the reference numbers of Table I.
QUINTANA, F. et al., “DNA fragments of the human 60-1 kDa heat shock protein (HSP60) vaccinate against adjuvant arthritis: identification of a regulatory HSP60 peptide”, J. Immunol., 171: 3533-3541 (2003).
BENAGIANO, M. et al., “Human 60-kDa heat shock protein 2 is a target autoantigen of T cells derived form atherosclerotic plaques”, J. Immunol., 174: 6509-6517, (2005).
RAZ, R. et al., “B-cell function in new-onset type diabetes 3 and immunomodulation with heat-shock protein peptide (DiaPep27): a randomised, double-blind, phase II trial”, The Lancet, 358:1749-1753 (2001).
FREESE, A. et al., “HLA-B7 B-pleated sheet-derived 4 synthetic peptides are immunodominant T-cell epitopes regulating alloresponces”, Blood, 99(9): 3286-3292 (2002).
GODKINS, A. et al., “Use of eluted peptide sequence data to 5 identify the binding characteristics of peptides to the insulin-dependent diabetes susceptibility allele HLA-DQ8 (DQ 3.2)”, Int. Immunol., 9(6): 905-911 (1997)
KOSMOPOULOU, A., “T-cell Epitopes of the La/SSB 6 Autoantigen: Prediction Based on the Homology Modeling of HLA-DQ2/DQ7 with the Insulin-B Peptide/HLA-DQ8 Complex”, J. Computational Chem., 27(9): 1033-1044 (2006)
SKIPPER, J. et al., Int. J. Canc. 82: 669 (1999). 7
LIU, G. et al., Canc. Res. 64: 4980 (2004) 8
PASS, H. et al., Canc. J. Sci Am. 4: 316 (1998) 9
KESSLER, J. H., J. Exp. Med. 193(1):73-88 (2001). 10
LIU, G., J. Immunother. 26(4):30′-12 (1997) 11
PEDERSEN, L. Ø., J. Investig. Dermatol. 118: 595-599 12 (2002)
U.S. Pat. No. 6,063,900 13
HERNANDEZ, J., Eur J Immunol, 34:2331-41 (2004) 14
DIAMOND, D. et al., Blood 90:1751-57 (1997) 15
BELYAKOV, I., Proc. Nat. Acad. Sci. USA, 95:1709 (1998) 16
U.S. Pat. No. 7,232,887 17

NÄSLUND, J. et al., Proc. Nat. Acad. Sci. USA, 91: 8378-18 8382 (1994)
GANDY, S., J. Clin. Invest. 115(5): 1121-1129 (2005) 19
BENNER, E. J. et al., PLoS ONE 3(1): el376 (2008) 20

The contents of any patents, patent applications, patent publications, or scientific articles referenced anywhere in this application are herein incorporated in their entirety.

### Sequence Listings

SEQ ID NO: 1
CD 20 - RITUXIMAB BINDING EPITOPE
CALMIANSC
SEQ ID NO: 2
CD20 - RITUXIMAB BINDING EPITOPE 2
CWWEWTIGC
SEQ ID NO: 3
HUMAN CD20 AMINO ACID SEQUENCE
MTTPRNSVNG TFPAEPMKGP IAMQSGPKPL FRRMSSLVGP
TQSFFMRESK TLGAVQIMNG LFHIALGGLL MIPAGIYAPI
CVTVWYPLWG GIMYIISGSL LAATEKNSRK CLVKGKMIMN
SLSLFAAISG MILSIMDILN IKISHFLKME SLNFIRAHTP
YINIYNCEPA NPSEKNSPST QYCYSIQSLF LGILSVMLIF
AFFQELVIAG IVENEWKRTC SRPKSNIVLL SAEEKKEQTI
EIKEEVVGLT ETSSQPKNEE DIEIIPIQEE EEEETETNFP
EPPQDQESSP IENDSSP
SEQ ID NO: 4
HUMAN CD25 (116-122) DACLIZUMAB BINDING EPITOPE
ERIYHFV
SEQ ID NO: 5
HUMAN CD25 ANALOG DACLIZUMAB BINDING EPITOPE
CWYHYIWEC

What is claimed is:

1. A process for manufacturing an antibody comprising using a composition comprising directed sequence polymers (DSPs) as an antigen, wherein the DSP composition is prepared in a method comprising the steps of:
A.(1) selecting a first base peptide sequence, wherein the sequence is an amino acid sequence of an epitope of interest;
A.(2) synthesizing by solid phase peptide synthesis a first cassette of the DSPs,
wherein, for each amino acid position of the first cassette of the directed sequence polymers, an amino acid is incorporated into a DSP, such amino acid randomly selected from a mixture of amino acids consisting of:
(i) an amino acid found at the corresponding position in said first peptide sequence, such amino acid present in the pool at a relative molar concentration of a0;
(ii) a primary replacement of the amino acid found at the said position in said selected amino acid sequence, said primary replacement defined according to amino acid similarity, such primary replacement amino acid present in the mixture at a relative molar concentration of a1;
(iii) a secondary replacement, if applicable, of the amino acid found at the said position in said selected amino acid sequence, said secondary replacement defined according to amino acid similarity, such secondary replacement amino acid present in the mixture at a relative molar concentration of a2;
(iv) a tertiary replacement, if applicable, of the amino acid found at the said position in said selected amino acid sequence, said tertiary replacement defined according to tertiary amino acid similarity, such tertiary replacement amino acid present in the mixture at a relative molar concentration of a3; and
(v) A: alanine, present in the mixture at a fixed relative molar concentration A,
wherein the amino acids in the mixture are present in a fixed molar input ratio relative to each other, determined prior to starting synthesis,
wherein the relative molar amount of A is more than 50% of the total amino acid concentration of the DSPs, and each of a0 and a1 is within the range of 0.05-50%, each of a2 and a3 is within the range of 0-50%, and wherein a0+a1+a2+a3=100−A;
A.(3) extending the length of the DSPs by
(a) repeating step (2) for 2 to 15 cycles and elongating the DSP under the same condition;
(b) repeating step (2) for 2 to 15 cycles and elongating the DSP, for each cycle, using a different input ratio of amino acids in the mixture;
(c) repeating steps (1) and (2) for 2 to 15 cycles and elongating the DSP using cassettes based on more than one base peptide; or
(d) assembling 2 to 15 cassettes synthesized in a single cycle of step (2); or
(e) assembling 2 to 15 cassettes, the first cassette synthesized under one condition of step (2), and second and more cassettes synthesized under a second condition of step (2);
A.(4) optionally further elongating the DSPs by repeating steps (2) and (3) for 2 to 15 cycles, wherein for each cycle a new cassette of the DSP is designed independently from the any of the previous cassettes designated by previous cycles of step (2);
wherein the number of cycles selected in steps (3) and (4) is selected so that the final length of the DSP is about 25 to 300 amino acid residues; and
B.(1) contacting the DSP with a means of generating antibodies;
B.(2) selecting a candidate antibody that bind to the DSP;
B.(3) identify the candidate antibody and determine a binding affinity of the candidate antibody to the first base peptide and further to a protein from which the first base peptide sequence was derived; and
B.(4) produce a useful quantity of the candidate antibody, thereby manufacturing an antibody.

2. The process for manufacturing an antibody according to claim 1, wherein the means of generating antibodies is a phage display library.

3. The process for manufacturing an antibody according to claim 1, wherein the means of generating antibodies is a B cell library.

4. The process for manufacturing an antibody according to claim 1 wherein the means of generating antibodies is a humanized cell library.

5. The process according to claim 1, wherein the amino acid sequence of the epitope is an epitope related to a cancer.

6. The process according to claim 5, wherein the epitope comprises a protein selected from G-protein coupled receptors (GPCR), CD20, vascular endothelial growth factor (VEGF), CD52, epidermal growth factor receptor (EGFR+), CD33, HER2.

7. The process according to claim 1, wherein the amino acid sequence of the epitope is an epitope related to TNF alpha, CD25 or immunoglobulin E, for immunosuppression, CD11a, alpha4-beta1 integrin; infectious disease related beta chemokine receptor CCR5, RSVgpP.

8. The process according to claim 6, wherein the amino acid sequence of the epitope is selected from the group consisting of SEQ ID NO: 1-2.

9. The process according to claim 1, wherein the amino acid sequence of the epitope is relevant to the pathology caused by or found concomitantly with the presence of an infectious disease agent.

10. The process according to claim 9, wherein the infectious disease agent is a virus causing or found concomitantly with a disease or condition selected from the group consisting of AIDS, AIDS Related Complex, Chickenpox (Varicella), Common cold, Cytomegalovirus Infection, Colorado tick fever, Dengue fever, Ebola haemorrhagic fever, Hand, foot and mouth disease, Hepatitis, Herpes simplex, Herpes zoster, HPV, Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease, and Yellow fever.

11. The process according to claim 9, wherein the infectious disease agent is a bacteria causing or found concomitantly with a disease or condition selected from the group consisting of Anthrax, Bacterial Meningitis, Botulism, Brucellosis, Campylobacteriosis, Cat Scratch Disease, Cholera, Diphtheria, Gonorrhea, Impetigo, Legionellosis, Leprosy (Hansen's Disease), Leptospirosis, Listeriosis, Lyme disease, Melioidosis, MRSA infection, Nocardiosis, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Typhus (including epidemic typhus), and Urinary Tract Infections.

12. The process according to claim 9, wherein the infectious disease agent is a parasite causing or found concomitantly with a disease or condition selected from the group consisting of Amoebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amoebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Plasmodium, Scabies, Schistosomiasis, Taeniasis, Toxocariasis, Toxoplasmosis, Trichinellosis, Trichinosis, Trichuriasis, Trichomoniasis, and Trypanosomiasis (including African trypanosomiasis).

13. The process according to claim 1, wherein the first base peptide sequence comprises two or more original sequences of one or more peptides which sequences were non-contiguous in the proteins, such original sequences made contiguous in the first base peptide sequence.

14. The process according to claim 13, wherein the original sequences were derived from more than two or more peptides.

15. A composition comprising an antibody manufactured by the process according to claim 1.

16. Use of a composition according to claim 15 for the manufacturer of a medicament for the treatment of a disease.

17. The composition of claim 15, wherein the antibody is a monoclonal antibody that recognizes a protein for multiple species.

18. The composition of claim 15 wherein the directed sequence peptides are modified post-synthesis.


SEQUENCE LISTING (85 sequences)

SEQ ID NO: 1 - protein, 9 aa, Homo sapiens
CALMIANSC

SEQ ID NO: 2 - protein, 9 aa, Homo sapiens
CWWEWTIGC

SEQ ID NO: 3 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 4 - protein, 7 aa, Homo sapiens
ERIYHFV

SEQ ID NO: 5 - protein, 9 aa, Homo sapiens
CWYHYIWEC

SEQ ID NO: 6 - protein, 20 aa, Homo sapiens
KFGADARALMLQGVDLLADA

SEQ ID NO: 7 - protein, 15 aa, Homo sapiens
LKVGLQVVAVKAPGF

SEQ ID NO: 8 - protein, 15 aa, Homo sapiens
GGAVFGEEGLTLNLE

SEQ ID NO: 9 - protein, 15 aa, Homo sapiens
TLNLEDVQPHDLGKV

SEQ ID NO: 10 - protein, 15 aa, Homo sapiens
VGAATEIEMKEKKDR

SEQ ID NO: 11 - protein, 15 aa, Homo sapiens
VGGTSDVEVNEKKDR

SEQ ID NO: 12 - protein, 15 aa, Homo sapiens
IVLGGGCALLRCIPA

SEQ ID NO: 13 - protein, 24 aa, Homo sapiens
VLGGGVALLRVIPALDSLTPANED

SEQ ID NO: 14 - protein, 15 aa, Homo sapiens
GCALLRCIPALDSLT

SEQ ID NO: 15 - protein, 15 aa, Homo sapiens
RCIPALDSLTPANED

SEQ ID NO: 16 - protein, 15 aa, Homo sapiens
EIIKRTLKIPAMTIA

SEQ ID NO: 17 - protein, 15 aa, Homo sapiens
VEKIMQSSSEVGYDA

SEQ ID NO: 18 - protein, 15 aa, Homo sapiens
MAGDFVNMVEKGIID

SEQ ID NO: 19 - protein, 15 aa, Homo sapiens
VNMVEKGIIDPTKVV

SEQ ID NO: 20 - protein, 15 aa, Homo sapiens
VAVTMGPKGRTVIIE

SEQ ID NO: 21 - protein, 15 aa, Homo sapiens
KGIIDPTKVVRTALL

SEQ ID NO: 22 - protein, 15 aa, Homo sapiens
PTKVVRTALLDAAGV

SEQ ID NO: 23 - protein, 15 aa, Homo sapiens
ASLLTTAEVVVTEIP

SEQ ID NO: 24 - protein, 9 aa, Homo sapiens
GETRKVKAH

SEQ ID NO: 25 - protein, 15 aa, Homo sapiens
RKVKAHSQTHRVDLG

SEQ ID NO: 26 - protein, 15 aa, Homo sapiens
RVDLGTLRGYYNQSE

SEQ ID NO: 27 - protein, 15 aa, Homo sapiens
DGRLLRGHDQYAYDG

SEQ ID NO: 28 - protein, 14 aa, Homo sapiens
GPEYWDRNTQIYKA

SEQ ID NO: 29 - protein, 16 aa, Homo sapiens
WDRNTQIYKAQAQTDR

SEQ ID NO: 30 - protein, 9 aa, Homo sapiens
RNTQIYKAQ

SEQ ID NO: 31 - protein, 15 aa, Homo sapiens
RESLRNLRGYYNQSE

SEQ ID NO: 32 - protein, 15 aa, Homo sapiens
GSHTLQSMYGCDVGP

SEQ ID NO: 33 - protein, 12 aa, Homo sapiens
LNEDLRSWTAAD

SEQ ID NO: 34 - protein, 15 aa, Homo sapiens
LNEDLRSWTAABTAA

SEQ ID NO: 35 - protein, 9 aa, Homo sapiens
DKGQVLNIQ

SEQ ID NO: 36 - protein, 14 aa, Homo sapiens
LEDKGQVLNIQMRR

SEQ ID NO: 37 - protein, 14 aa, Homo sapiens
AFKGSIFVVFDSIE

SEQ ID NO: 38 - protein, 9 aa, Homo sapiens
ESAKKFVET

SEQ ID NO: 39 - protein, 14 aa, Homo sapiens
IESAKKFVETPGQK

SEQ ID NO: 40 - protein, 12 aa, Homo sapiens
AKDANNGNLQLR

SEQ ID NO: 41 - protein, 9 aa, Homo sapiens
EALKKIIED

SEQ ID NO: 42 - protein, 9 aa, Homo sapiens
EQIKLDEGW

SEQ ID NO: 43 - protein, 12 aa, Homo sapiens
LKEQIKLDEGWV

SEQ ID NO: 44 - protein, 9 aa, Homo sapiens
AELMEISED

SEQ ID NO: 45 - protein, 13 aa, Homo sapiens
SKAELMEISEDKT

SEQ ID NO: 46 - protein, 9 aa, Homo sapiens
KGSIFVVFD

SEQ ID NO: 47 - protein, 14 aa, Homo sapiens
AKDANNGNLQLRNK

SEQ ID NO: 48 - protein, 9 aa, Homo sapiens
DANNGNLQL

SEQ ID NO: 49 - protein, 12 aa, Homo sapiens
IVEALSKSKAEL

SEQ ID NO: 50 - protein, 13 aa, Homo sapiens
AFKGSIFVVFDSI

SEQ ID NO: 51 - protein, 14 aa, Homo sapiens
GSIFVVFDSIESAK

SEQ ID NO: 52 - protein, 14 aa, Homo sapiens
IFVVFDSIESAKKF

SEQ ID NO: 53 - protein, 9 aa, Homo sapiens
VVFDSIESA

SEQ ID NO: 54 - protein, 13 aa, Homo sapiens
ELMEISEDKTKIR

SEQ ID NO: 55 - protein, 9 aa, Homo sapiens
EALYLVCGE

SEQ ID NO: 56 - protein, 9 aa, Homo sapiens
KTWGQYWQV

SEQ ID NO: 57 - protein, 10 aa, Homo sapiens
KTWGQYWQVL

SEQ ID NO: 58 - protein, 9 aa, Homo sapiens
ITDQVPFSV

SEQ ID NO: 59 - protein, 10 aa, Homo sapiens
TITDQVPFSV

SEQ ID NO: 60 - protein, 10 aa, Homo sapiens
LLDGTATLRL

SEQ ID NO: 61 - protein, 10 aa, Homo sapiens
VLYRYGSFSV

SEQ ID NO: 62 - protein, 9 aa, Homo sapiens
VLKRCLLHL

SEQ ID NO: 63 - protein, 10 aa, Homo sapiens
ALDGGNKHFL

SEQ ID NO: 64 - protein, 10 aa, Homo sapiens
VLPSPACQLV

SEQ ID NO: 65 - protein, 9 aa, Homo sapiens
YLEPGPVTA

SEQ ID NO: 66 - protein, 10 aa, Homo sapiens
SLADTNSLAV

SEQ ID NO: 67 - protein, 9 aa, Homo sapiens
SVSVSQLRA

SEQ ID NO: 68 - protein, 9 aa, Homo sapiens
LNVSLADTN

SEQ ID NO: 69 - protein, 10 aa, Homo sapiens
SLYSFPEPEA

SEQ ID NO: 70 - protein, 9 aa, Homo sapiens
SVYDFFVWL

SEQ ID NO: 71 - protein, 10 aa, Homo sapiens
ELAGIGILTV

SEQ ID NO: 72 - protein, 9 aa, Homo sapiens
AAGIGILTV

SEQ ID NO: 73 - protein, 10 aa, Homo sapiens
EAAGIGILTV

SEQ ID NO: 74 - protein, 10 aa, Homo sapiens
AAGIGILTVI

SEQ ID NO: 75 - protein, 9 aa, Homo sapiens
KMVELVHFL

SEQ ID NO: 76 - protein, 9 aa, Homo sapiens
RLFFYRKSV

SEQ ID NO: 77 - protein, 10 aa, Human herpesvirus 5
ILARNLVPMV

SEQ ID NO: 78 - protein, 9 aa, Human herpesvirus 5
ELEGVWQPA

SEQ ID NO: 79 - protein, 9 aa, Human herpesvirus 5
RIFAELEGV

SEQ ID NO: 80 - protein, 9 aa, Human herpesvirus 5
NLVPMVATV

SEQ ID NO: 81 - protein, 15 aa, Human immunodeficiency virus 1
RIQRGPGRAFVTIGK

SEQ ID NO: 82 - protein, 13 aa, Artificial Sequence
  Description of Artificial Sequence Synthetic peptide
AKAVAAWTLKAAA

SEQ ID NO: 83 - protein, 5 aa, Artificial Sequence
  Description of Artificial Sequence Synthetic peptide
THMCE

SEQ ID NO: 84 - protein, 5 aa, Artificial Sequence
  Description of Artificial Sequence Synthetic peptide
PWKNA

SEQ ID NO: 85 - protein, 4 aa, Artificial Sequence
  Description of Artificial Sequence Synthetic peptide
RGDS